(19) United States
(12) Patent Application Publication
(10) Pub. No.: US 2010/0279381 A1
(43) Pub. Date: Nov. 4, 2010

(54) Acetylcholinesterase (AChE) variants of the N-terminus

(75) Inventors: Hermona Soreq, Jerusalem (IL); Eran Meshorer, Rockville, MD (US)

Correspondence Address: BROWDY AND NEIMARK, P.L.L.C.;624 NINTH STREET, NW, SUITE 300, WASHINGTON, DC, 20001-5303, US

(73) Assignee: Yissum Research Development Company of the Hebrew University of Jerusalem, Jerusalem (IL)

(21) Appl. No.: 11/546,545

(22) Filed: Oct. 12, 2006

Related U.S. Application Data
(63) Continuation-in-part of application No. PCT/IL05/00388, filed on Apr. 13, 2005.

(30) Foreign Application Priority Data
Apr. 13, 2004 (IL) 161354

(51) Int. Cl. C12N 9/18; C07H 21/04; C12N 5/07

(52) U.S. Cl. 435/197; 536/23.1; 435/325

(57) ABSTRACT

A novel form of acetylcholinesterase (AChE) is provided, N-AChE, which bears a transmembrane domain. Exons encoding this novel form, the peptide comprising, the transmembrane domain, as well as antibodies recognizing the same are also provided. N-AChE expression in the hippocampus is correlated with Alzheimer's disease. Secreted forms of AChE are also provided, and methods of producing AChE protein are, also described.

### STATEMENT AS TO FEDERALLY SPONSORED RESEARCH

This work was supported by the US Army Medical Research and Material Command DAMD 17-99-9547 (July 1999-August 2004). The US Government has certain rights in this invention.

### FIELD OF THE INVENTION

The present invention relates to the field of cholinergic signaling. More specifically, the present invention refers to novel variants of acetylcholinesterase (AChE).

### BACKGROUND OF THE INVENTION

All publications mentioned throughout this application are fully incorporated herein by reference, including all references cited therein.

Acetylcholinesterase (AChE) terminates synaptic transmission by hydrolyzing the neurotransmitter acetylcholine at cholinergic synapses [Massoulie, J. (2002) Neurosignals 11, 130-143]. At least three different mRNAs, with distinct 3′ regions are produced by alternative splicing from the unique ACHE gene present in vertebrates [Soreq, H., and Seidman, S. (2001) Nat Rev Neurosci 2, 294-302]. These encode for AChE isoforms with different C-termini responsible for distinct cell adherence and non-catalytic properties: the ‘synaptic’, AChE-S (a.k.a. ‘tailed’ AChE-T), the ‘erythrocytic’ AChE-E (a.k.a. ‘hydrophobic’, AChE-H) and the ‘readthrough’, AChE-R. AChE-S mRNA is ubiquitously expressed and is subject to transcriptional and post-transcriptional development-related regulation [Coleman and Taylor, (1996) J. Biol. Chem. 271(8): 4410-6; Fuentes and Taylor (1993) Neuron 10(4): 679-87; Rotundo et al. (1998) J. Physiol. Paris. 92(3-4): 195-8]. AChE-R is the isoform induced by stress, rarely found in adult tissues under basal conditions [Meshorer, E. et al. (2002) Science 295, 508-512], and AChE-E is primarily expressed in red blood cell progenitors [Chan et al. (1998) J. Biol. Chem. 273(16): 9727-33].

The ACHE gene displays a complex expression pattern, not restricted to cholinergic or even nervous system tissues. Rather, it extends to non-cholinergic, non-cholinoceptive tissues including retinal pigmented epithelium [Martelly and Gautron (1988) Brain Res. 460(2):205-13], spleen [Bellinger et al. (1993) Brain Res. Bull. 32(5): 549-54] and liver [Satler et al. (1974) Histochemistry. 39(1):65-70], to name a few. This led to the working hypothesis that the AChE protein might have additional roles. Several non-enzymatic activities have been demonstrated, including neuritogenesis [Grifman M. et al. (1998) Proc. Natl. Acad. Sci. USA. 95: 13935-13940], muscle development [Behra et al. (2002) Nat. Neurosci. 5(2):111-8], cell-cell interaction [Darboux et al. (1996) EMBO J. 15(18): 4835-43], facilitation of beta-amyloid peptide assembly into Alzheimer's fibrils [Inestrosa, N. et al. (1996) Neuron. 16: 881-891; Rees et al., (2003) Neurobiol Aging. 24(6):777-87], hematopoiesis [Paoletti, F. et al. (1992) Blood. 79(11): 2873-2879; Grisaru, D. et al. (2001) Molecular Medicine 7(2): 93-105], and apoptosis [Zhang et al. (2002) Cell Death Differ. 9(8):790-800].

Although most of the efforts for understanding ACHE gene organization focused on the 3′ end of AChE mRNA, the 5′ end as well attracted attention. The ACHE promoter region of the mouse, rat and human genes were studied [Mutero, A. et al. (1995) J Biol Chem 270, 1866-1872; Chan et al. (1999) Proc Natl Acad Sci USA. 96(8):4627-32; Getman et al., (1995) J Biol Chem. 270(40):23511-9]. In mouse, five E-boxes and a GC-rich sequence that contains binding sites for the Sp1 and Egr-1 transcription factors were identified in the upstream region of ACHE [Mutero (1995) id ibid.]. These binding sites were particularly important for the response to muscarinic acetylcholine receptor activation (von der Kammer et al., 1998). A second promoter, located approximately 2 kb upstream from the transcription start site in exon 2, has been reported in the mouse ACHE locus [Atanasova, E. et al. (1999) J Biol Chem 274, 21078-21084]. In the human ACHE gene, GC-rich sequences were identified upstream to the cap site, containing functional binding sites for Sp1, Egr-1 and AP2 [Getman (1995) id ibid.]. More recently, a 22 kb region located upstream of the human ACHE was sequenced and analyzed [Grisaru et al. (1999) Mol Cell Biol. 19(1):788-95; Shapira, M. et al. (2000) Hum Mol Genet 9, 1273-1281]. Several clusters of binding sites for osteogenic transcription factors, e.g. Krox-20/Egr-2, vitamin D receptor and estrogen receptor were identified [Grisaru (1999) id ibid.]. In addition, a 4 by deletion associated with intensified expression and increased hypersensitivity to anti-cholinesterases was found ca. 17 kb upstream to the transcription start site. Interestingly, this deletion disrupts a glucocorticoid responsive element (GRE) [Shapira (2000) id ibid.]. Finally, in the rat, a muscle-specific enhancer was identified within the first intron of ACHE, which contains an N-box motif essential for AChE expression in skeletal muscle fibers [Chan (1999) id ibid.].

To better understand the regulation of the ACHE gene in response to stress, the inventors investigated its promoter organization combining in silico and molecular biology approaches. Various novel 5′ alternative transcripts were identified in both mouse and human ACHE genes, amongst which one encoding a novel human membranal AChE protein variant with an extended N-terminus. In the present study, the inventors report their tissue and cell type distributions and regulation by stress and the glucocorticoid receptor (GR), and describe the organization of the corresponding promoters.

Furthermore, the inventors investigated the expression of the novel 5′ alternative transcript, as well as its protein product (an AChE molecule with an N-terminal transmembrane domain) in hippocampus of Alzheimer's disease specimens.

The expression of the novel N-AChE in Alzheimer's hippocampus, together with the finding that overexpression of different forms of AChE can alter gene expression in neuronal lineage cells, especially of genes involved in splicing and apoptotic events reinforce the hypothesis of a causal relationship between AChE and Alzheimer's disease.

Thus, it is an object of the present invention to provide novel AChE cDNA variants, which differ at their 5′ end. Consequently, the present invention also provides novel AChE proteins, with an extended N-terminus, as well as novel human and mouse peptides consisting of the novel AChE N-terminus. An antibody which specifically recognizes the novel N-AChE protein is also provided, as well as its use in diagnostic procedures. Other uses and objects of the invention will become clear as the description proceeds.

### SUMMARY OF THE INVENTION

In a first aspect, the present invention provides a cDNA sequence derived from the ACHE gene, comprising a variant 5′ region, wherein said ACHE gene may be from mouse or human origin.

In other words, the present invention presents a cDNA sequence comprising an AChE variant at its 5′ end. Said variant sequence is substantially as denoted by any one of SEQ. ID. Nos.1, 2, 3, 4, 5, 6, 7, 8, 9 and 10 (see FIG. 1 and Table 3), as well as functional analogues and derivatives thereof.

In a second aspect, the present invention provides a peptide encoded by a nucleic acid sequence derived from the ACHE gene, wherein said peptide comprises AChE transmembrane and intracellular domains, and said ACHE gene may be from mouse or human origin.

In one embodiment, said peptide is denoted by any one of SEQ. ID. Nos.11 and 12 (see FIG. 6 and Table 3), as well as functional analogues and derivatives thereof.

In another embodiment, said peptide is derived from the human ACHE gene, and comprises the sequence substantially as denoted by any one of SEQ. ID. Nos.12, 13 and 14 (see Table 3), as well as functional analogues and derivatives thereof.

In a further embodiment, said peptide is derived from the mouse ACHE gene, and comprises the sequence denoted by SEQ. ID. No.11 (see Table 3), as well as functional analogues and derivatives thereof.

In a yet further embodiment, the present invention provides a peptide derived from a novel human AChE transmembrane and intracellular domain, wherein said peptide is substantially as denoted by any one of SEQ. ID. Nos.13 and 14 (see Table 3), as well as functional analogues and derivatives thereof.

In a third aspect, the present invention provides an AChE protein comprising a transmembrane domain. Thus, the novel AChE protein is comprised of an extracellular, a transmembrane and an intracellular domain.

In one embodiment, said novel AChE protein may be of the -S, -R or -E forms, denoted by sequences SEQ. ID. Nos.15, 16 and 17 (see Table 3 and FIG. 4), respectively, as well as functional analogues or derivatives thereof.

In another aspect, the present invention provides a nucleic acid construct comprising any one of the sequences denoted by SEQ. ID. Nos.1-10 and 36-38, operably linked to at least one control element.

In one embodiment said construct may be an expression vector. In a further aspect, the present invention provides a transfected cell containing an exogenous sequence, wherein said cell is transfected with the construct of the invention, or with any one of the sequences corresponding to the novel 5′ AChE variants described herein.

Hence, in an even further aspect, the present invention provides a marker for any one of stress, cholinergic balance and Alzheimer's disease, wherein said marker consists of an AChE mRNA comprising a variant 5′ region. The glucocorticoid and stress dependence of the new exons suggests the use of such markers to identify hormone and stress-induced diseases.

In one embodiment, said variant 5′ region is essentially as denoted by any one of SEQ. ID. Nos. 3, 4 and 5 (see Table 3), as well as functional analogues and derivatives thereof.

In another embodiment, said marker is not responsive to cortisol treatment, and said variant 5′ region is essentially as denoted by SEQ. ID. No. 3, as well as functional analogues and derivatives thereof.

In a further embodiment, said marker is responsive to cortisol treatment, and said variant 5′ region is essentially as denoted by any one of SEQ. ID. Nos. 4 and 5, as well as functional analogues and derivatives thereof.

Thus, in a further aspect, the present invention provides an antibody recognizing an N-terminal AChE intracellular domain. Said antibody is directed against a synthetic peptide essentially as denoted by any one of SEQ. ID. Nos.13 and 14 (see Table 3 and FIG. 4), as well as any variants, fragments or derivatives thereof.

The present invention also provides a pharmaceutical composition comprising as active agent the anti-N-AChE antibody as defined above.

Further, the present invention provides the use of anti-AChEs, as well as the above-described antibody for intracellular signaling in cells expressing the AChE transmembrane domain (denoted by SEQ. ID. No.34). Said antibody, and inhibitors, may also be used as a ligand for AChE. Therefore, cells expressing this variant may serve as extremely sensitive biosensors, which would respond to binding of inhibitors or antibodies, by modifying intracellular signaling, through the kinase binding domain of N-AChE. In this respect, another aspect provided by the present invention is a sensor for a cholinergic signal, wherein said sensor comprises the AChE extracellular, transmembrane and intracellular domains, denoted by any one of SEQ. ID. Nos. 11 and 12 (Table 3).

In a different aspect, the sensor of stress and cholinergic imbalance may be provided by the use of a cell expressing an AChE transmembrane domain, wherein said transmembrane domain is as described above.

In a yet further aspect, the present invention also provides a plurality of sensors for cholinergic signaling, embedded in (or affixed to) a suitable solid matrix. These sensors, when blocked with organophosphates or any anti-cholinesterases, will send a signal which would activate the kinase binding domain in the intracellular region of N-AChE and induce a signal transduction cascade which would be selective for this N-AChE variant alone. The fact that the novel variants were detected in different lymphoid lineages at specific stages of development, as shown in FIG. 4C, suggested that these novel variants may be a marker for lymphoid cell lineage differentiation, wherein said marker comprises the sequence substantially as denoted by any one of SEQ. ID. Nos.11 and 12 (see Table 3), as well as any fragments, derivatives and analogues thereof, and wherein a decrease in the level of its expression denotes a more advanced stage of lymphoid differentiation.

One additional aspect of the invention relates to a method for diagnosis of Alzheimer's disease, comprising administering the antibody described in the invention, which recognizes the novel variant N-AChE, labeled with a detectable marker, to the subject to be diagnosed, and detecting the presence of the antibody in the hippocampus through imaging techniques.

In a further aspect, the present invention provides an AChE protein, wherein said protein is denoted by one of sequences SEQ. ID. Nos. 15, 16 and 17, as well as derivatives thereof, wherein said protein is secreted.

The present invention also provides a secreted AChE protein, wherein said protein comprises at its N-terminus the sequence denoted by SEQ. ID. No.39.

Another AChE protein provided by the present invention is a derivative of AChE comprising a transmembrane domain and/or an intracellular domain, wherein one or both said domains has at least one deleted, inserted or substituted residue, and said AChE protein is secreted.

Finally, the present invention provides a method of recombinantly producing an AChE protein, said method comprising preparing a culture of recombinant host cells transformed or transfected with a recombinant nucleic acid molecule encoding an AChE protein or with an expression vector comprising said recombinant nucleic acid molecule; culturing said host cell culture under conditions permitting the expression of said protein; and recovering said protein from the cells. Preferably, said nucleic acid molecule is denoted by one of SEQ. ID. No. 36, SEQ. ID. No. 37 and SEQ. ID. No.38.

### BRIEF DESCRIPTION OF THE FIGURES

FIGS. 1A-1D: Mouse and human 5′ genomic region and 5′ transcripts.

FIG. 1A: Shown are 2.6 kb of the 5′ genomic region of the mouse ACHE gene. Exons (shaded gray or underlined) are named on the right. Splice sites are shown in yellow, translation start sites in red. The bottom line shows the beginning of exon 2.

FIG. 1B: Schematic representation of the entire 5′ region of the ACHE gene containing the variant exons. All schemes are drawn to scale. Exons verified by sequencing are painted aquamarine and are connected by straight lines. Non-validated, in brackets, is white and connected by a dashed red line. The long cDNA clone (AK036443, mE1c-long) is shown in gray. The ORF of mE1e is red, the one in E2 is orange. Abbreviations: Conf., confirmed; evid., evidence; N.-val., non-validated; cony., conventional; nov., novel.

FIGS. 1C-D: The 2.65 kb of the 5′ region of the human ACHE gene and the corresponding scheme. The two possible starting ATGs for hE1d are shown in pink and red. The second ATG corresponds to mE1e's ATG.

FIGS. 2A-2D: Promoter and syntheny analyses of mouse and human ACHE genes.

FIG. 2A: Cister software analysis for 7.1 kb of mouse (top) and human (bottom) ACHE genes, including 3.55 kb of upstream sequence and 3.55 kb of the coding region, representing the overall probability for a specific region to function as a promoter. Colored lines represent selected transcription factor binding sites, detailed below. Red triangles represent putative glucocorticoid response elements (GREs). The different alternative 5′ exons (gray boxes) are marked a-e for mouse and a-d for human. Base counts from the starting ATG (+1) are marked above (dashed lines). For comparison, the human sequence was analyzed with the Chip2Promoter software (Genomatix suite). Human promoter predictions are shown as orange boxes (hP1, hP2 and hP3), gene-associated promoter (hP2, defined by the program as the proximal promoter to the first exon) is shown as a yellow box. Chip2Promoter does not support the mouse sequence, so the promoter regions were determined according to Cister, shown as empty brick-colored boxes (mP1, mP2 and mP3, top). Abbreviations: Se. bind.si., selected binding sites.

FIG. 2B: MatInspector analysis of the predicted binding sites for transcription factors. Factors have been grouped according to structure, function, motif recognition or others, depicted by different colors and shapes shown on the left. Blast-2-sequences analysis (www.ncbi.nlm.nih.gov/blast) of the 5′ region of mouse (top) vs. the human (bottom) ACHE. Homologous sequences are depicted as color-matched boxes. Exons are shown as empty boxes below.

FIGS. 2C-2D: SINEs and LINEs distribution in the upstream regions of mouse (Mo., 9.5 kb, top) and human (Hu., 20 kb, bottom) genes, screened for SINEs (blue circles) and LINEs (green circles). The distal ACHE promoter [Shapira (2000) id ibid.] is shown in red. Repeat counts for 500 by (Rep./500 bp) are shown in D for both mouse (top) and human (bottom).

FIGS. 3A-3B: Tissue and cell type expression patterns of AChE's alternatively spliced transcripts.

FIG. 3A: RT-PCR products and their corresponding molecular sizes (right) of the 5′ (four upper lanes: mE1a, mE1b, mE1c and mE1d) and 3′ (three lower lanes: AChE-S, AChE-R and AChE-R) alternative transcripts of murine AChE. Primer positions for each transcript are depicted on the left diagram (triangles) (for primer sequences, see Materials and Methods). Abbreviations: he., heart; mu., muscle; te., testis; ki., kidney; ap. Co., spinal cord; liv., liver; spl., spleen; thy., thymus; int., intestine; bas. Nu., basal nuclei; PFC, prefrontal cortex; hipp, hippocampus; cort., cortex; br. St., brain stem.

FIG. 3B: Representative fluorescent images of transcripts including mE1a, mE1b and mE1d in PFC (I), hipp (II) and cerebellum (cer, III) of naïve FVB/N mice. Cartoons on the right show the enlarged areas (red boxes). Enlargement of a cerebellar area (boxed) shows strong cytoplasmic labeling of mE1a (IV) and cytoplasmic and nuclear labeling of mE1d (V) in Purkinje cells. An enlargement of a single Purkinje cell with a labeled axon is shown on the bottom right panel (VI), with a schematic drawing on the right. Bars=50 μm. Abbrebiations: ce. bo., cell body; dend., dendrites; ax., axon.

FIGS. 4A-4G: Human embryonic expression of hN-AChE.

FIG. 4A: FISH detection of hE1d mRNA in sections from 16 (left), 24 (middle) and 34 (right) weeks old human embryonic brain (br., top) and thymus (thy., bottom). Bar graphs on the right show increased fractions with development of labeled cells (lab. ce.; *, P<0.05; ***, P<0.0005; 2-tailed Student's t-test).

FIG. 4B: AChE protein composition and epitope locations of the antibodies used (N, N-terminus; SP, signal peptide; Core, AChE core domain). The three different optional C-termini are depicted on the right. Inset: hE1d expression in T cells leukemia.

FIGS. 4C-4F: Hematopoietic expression of membranal hN-AChE.

FIG. 4C: Four distinct cell populations were distinguished by flow cytometry, using CD45 detection vs. side scatter plot (M, monocytes; G, granulocytes; P, progenitors; L, lymphocytes).

FIG. 4D: hN-AChE labeling (purple) was compared to an isotype control (green) demonstrating its expression in monocytes (Mon.), granulocytes (Gran.), lymphocytes (lymp.) and blood cell progenitors (prog.), to a lesser extent. No increases were observed following permeabilization of the cells (right), indicating membranal expression. Abbreviations: bd. Perme., before permeabilization; aft. Perme., after permeabilization.

FIG. 4E: FACS separation of cell populations.

FIG. 4F: Percent positive (pos.) cells before (−) and after permeabilization (+) of the noted CD45+ cell lineages. Average of 4 different cord blood preparations.

FIG. 4G: Lymphocyte sub-classification. Specific markers (CD34, stem cells; IL7, early lymphocytes; CD3, mature T-lymphocytes; CD19, mature B-lymphocytes) demonstrate elevated hN-AChE expression in mature T lymphocytes. Pos.=positive.

FIG. 5: Stress and glucocorticoid-related regulation of murine 5′ alternative exons.

Shown is RT-PCR analysis of mE1b, mE1c, mE1d, mAChE-S and actin in the cortices of neuron-specific glucocorticoid-receptor (GR) knockout (GR^{NesCre}) and wild-type (wt) mice 2 hr following 30 min of immobilization stress. Note that mE1b and mAChE-S were down-regulated following stress in GR^{NesCre} but not in wt mice. Exon mE1c was over-expressed following stress regardless of the presence (wt) or absence (GR^{NesCre}) of the GR. mE1d, as well, was over-expressed following stress, but only faintly detected in GR^{NesCre} mice, as compared to wt, attesting to its glucocorticoid-dependence. Actin mRNA served as control. Quantifications (against actin levels) are shown on the right (average of 3 animals in each group). Stars note statistically significant differences from controls. Na.=naïve; str.=stress.

FIGS. 6A-6E: N-AChE protein.

FIG. 6A: DNA sequence homology between mE1e (top) and hE1d (bottom). Total similarity is 79%. The in-frame ATGs are colored.

FIG. 6B: Amino acid sequence of mN-AChE (mE1e) (top) and hN-AChE (hE1d) (bottom). Identical amino acids are boxed, related amino acids are lined. Hydrophobic amino acids are red, positively charged amino acids are blue (arginine and lysine, dark blue; histidine, light blue). Putative phosphorylation sites are green; putative N-myristoylation sites are dark yellow. The last methionine is the translation start site on exon 2. (analysis used GENESTREAM, http://vega.igh.cnrs.fr/bin/align-guess.cgi). Secondary structure prediction (GOR4 software, http://npsa-pbil.ibcp.fr/cgi-bin/secpred_gor4.pl) is depicted above and below each sequence (c=random coil, e=extended strand, h=alpha helix). Note the lack of alpha helices and beta sheets of hN-AChE.

FIG. 6C: Expression in human brain regions. Inset, top left: Extracts of cultured human glioblastoma cells. Note similarity of labeling patterns for anti-hN-AChE and anti-core-AChE antibody (N19, Santa Cruz Biotechnology). Center: hN-AChE in different human brain regions. Note prominent hN-AChE expression in the occipital cortex (oxc), and significant labeling in hippocampus (hipp), prefrontal cortex (PFC), cortex, striatum (str) and amygdala (amg). Very weak bands were observed in the cerebellum (cereb).

FIG. 6D: FISH: hE1d mRNA probe labels both cell bodies and neurites of neurons in adult human PFC.

FIG. 6E: Locations of the different brain regions tested. See abbreviations in legend for FIG. 6C.

FIGS. 7A-7C: Predicted combinatorial complexity of the 5′ and 3′ AChE mRNA variants and their protein products. Shown are the

FIG. 7A: Splice and regulation patterns of the putative mouse ACHE transcripts.

FIG. 7B: Predicted promoters (prom.) of the putative mouse ACHE transcripts.

FIG. 7C: Predicted protein products of the putative mouse ACHE transcripts. Arrows note enhancing stimuli (GC=glucocorticoids). Doubly induced (doub.-ind.) variants (var., mE1c-R, mE1d-R) include both 5′ and 3′ exons which respond to GCs and stress. Extended N-AChE proteins may have one or more transmembrane domains at their N terminus Str.=stress.

FIG. 8: Schematic illustration of the human hippocampus showing main hippocampal regions in which levels and localization of AChE variants were studied. Abbreviations: Amyg., amygdale; Hipp. Form., hippocampal formation; form. & mamm. Bo., formix and mammillary body; S.c.p., Schaffer collateral pathway; M.f.p. Mossy fiber pathway; D.g., dentate gyrus; P.p., perforant pathway.

FIGS. 9A-9B: Downregulation of AChE expression in dentate gyrus neurons of Alzheimer's disease brain.

FIG. 9A: Immunohistological staining of control and Alzheimer's disease (AD) brain, using an antibody against the core domain of AChE, reveals massive downregulation of total AChE levels in dentate gyrus neurons. Top-Schematic representing the AChE protein and the region recognized by the antibody.

FIG. 9B: Histogram graph showing the quantification of the results presented in FIG. 9A. Arb.u.=arbitrary units.

FIG. 10: Changes in the expression of the AChE-S and AChE-R transcripts in the dentate gyrus of AD brain.

FIG. 10A: Photomicrograph of FISH staining of dentate gyrus from control (left) and AD (right) human hippocampus, using a probe specific to AChE-S transcript. Top-Schematic of AChE gene, arrow pointing the specificity of the probes.

FIG. 10B: Photomicrograph of FISH staining of dentate gyrus from control (left) and AD (right) human hippocampus, using a probe specific to AChE-R transcript.

FIG. 10C: Histogram graph showing the quantification of the results presented in FIGS. 10A and 10B. (*p<0.01, **p<0.05 Student's t-test) mRNA exp.=mRNA expression.

FIGS. 11A-11C: N-AChE is expressed in dentate gyrus of AD human brain.

FIG. 11A: Photomicrograph of FISH staining of dentate gyrus from control (left) and AD (right) human hippocampus, using an E1b-specific probe. Top-Schematic of AChE gene, arrow pointing the specificity of the probe.

FIG. 11B: Photomicrograph of FISH staining of CA3 neurons from control and AD human hippocampus, using an E1b-specific probe.

FIG. 11C: Histogram graph showing the quantification of the results presented in FIGS. 11A and 11B.

*p<0.01, lines indicate 50 mm and 10 mm in micrographs and insets respectively. mRNA exp.=mRNA expression.

FIGS. 12A-12B: Upregulation of the N-AChE-S variant in the mossy fiber system of AD human brain.

FIG. 12A: Immunohistochemistry of the mossy fiber system, of control (CT) and AD brains, with an antibody specific to the novel N′ terminus. Top-Schematic representing the N-AChE protein and the region recognized by the antibodies.

FIG. 12B: Immunohistochemistry of the mossy fiber system of control (CT) and AD brains, with an antibody specific to the C′ terminus. Bottom-Representation of the decreasing antibody concentration (Ab. Conc.) used in 12A and 12B.

FIG. 13: AChE transcripts are expressed in human AD hippocampus.

Left: Schematic diagram of the AChE gene. Arrows represent primers used in the RT-PCR reaction.
Right: Gel electrophoresis of RT-PCR of human AD hippocampus, confirming the expression of all AChE transcripts (AChE-Eld, AChE-R, AChE-S). T. AChE=Total AChE

FIG. 14: Schematic of the human hippocampus, showing AChE staining in AD specimens.

Abbreviations: NFT, neurofibrillary tangles; T. AChE assoc. w. NFTs+plaq., total AChE associated with NFTs and plaques; Mfp, mossy fiber pathway.

FIG. 15: Pie diagram showing the fraction of each functional group of genes among the total population of probes in the microarray. This figure shows that the composition of the chip is as follows:
17% snRNPs;
8% hnRNPs;
9% SR and SR related;
5% helicases (spliceosome associated);
6% spliceosome assembly mediators (splic. ass.med.);
8% splicing factor phosphorylation (Splic. fac. phos.);
6% other mRNA processing (e.g. polyadenylation, export);
11% targets (genes undergoing alternative splicing);
8% other spliceosomal components;
16% apoptosis-related genes undergoing alternative splicing;
5% other genes (oth. ge.);
1% unknown function (unk. fun.).

FIGS. 16A-16C: Results of the microarray analysis—Total population of transcripts on the array.

FIG. 16A: Histogram representing genes expressed in control versus AChE-S-treated cells.

FIG. 16B: Histogram representing genes expressed in control versus AChE-R-treated cells.

FIG. 16C: Graph showing the log ratio of the results in 16A and 16B. Abbreivations: cont., control, cum. dist. func., cumulative distribution function, rat., ratio.

FIGS. 17A-17I: Results of the microarray analysis, in histograms.

FIG. 17A: Photograph of the microarray.

FIG. 17B: Comparison of transcripts of target genes under AChE-R versus AChE-S treatment.

FIG. 17C: Comparison of transcripts of SR and SR-related genes under AChE-R versus AChE-S treatment.

FIG. 17D: Comparison of transcripts of house-keeping genes (HKG) under AChE-R versus AChE-S treatment.

FIG. 17E: Comparison of transcripts of mRNA processing genes under AChE-R versus AChE-S treatment.

FIG. 17F: Comparison of transcripts of splicing factor phosphorylation genes under AChE-R versus AChE-S treatment.

FIG. 17G: Comparison of transcripts of apoptosis genes under AChE-R versus AChE-S treatment.

FIG. 17H: Comparison of transcripts of spliceosomal component genes under AChE-R versus AChE-S treatment.

FIG. 17I: Comparison of transcripts of other categories of genes under AChE-R versus AChE-S treatment.

### DETAILED DESCRIPTION OF THE INVENTION

In the present study the inventors demonstrate that human and mouse ACHE genes contain at least four alternative first exons each, of which at least one encodes for an extended N-terminus. The extended AChE protein was named hN-AChE, and it was found to be expressed in the nervous system and blood cells, during various stages of their development.

The alternative novel first AChE exons display expression profiles distinct from those of the 3′ exons, which were described previously [Soreq and Seidman (2001) id ibid.] This rules out the possibility of a particular first exon being strictly associated with a given 3′ exon. The 3′ splicing options of the murine and human AChEs (AChE-S, AChE-R, AChE-E) may thus yield up to 15 and 12 different mRNA transcripts, respectively.

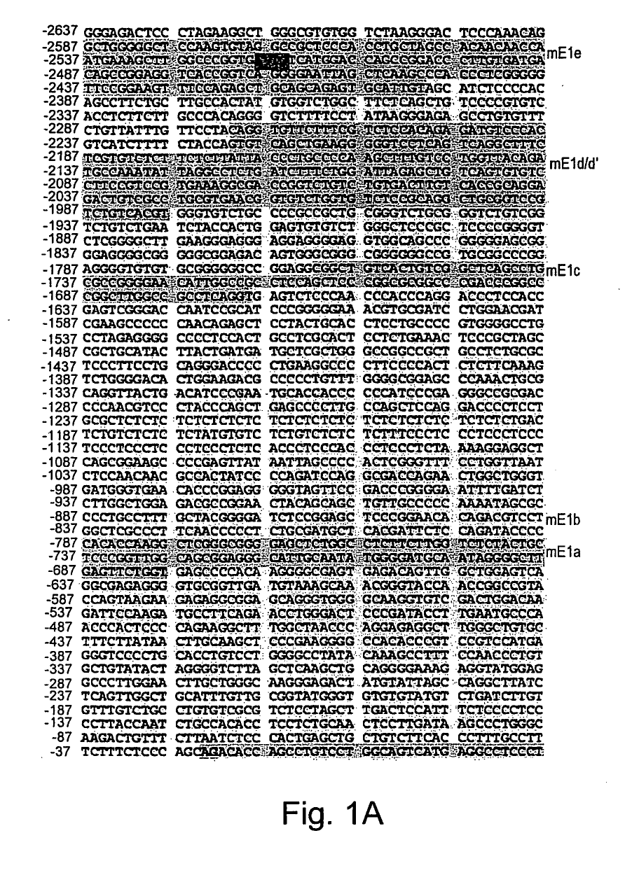

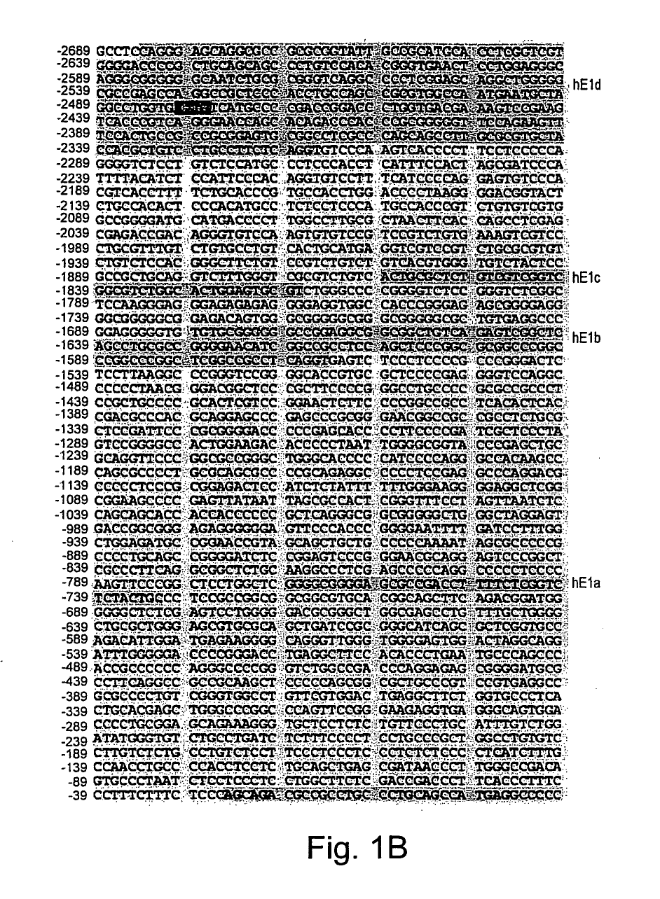

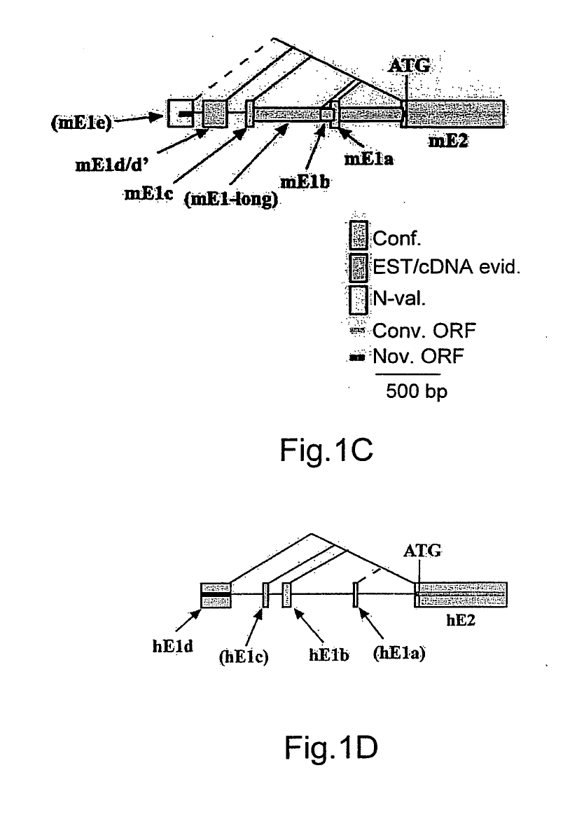

In other words, the present invention presents a cDNA sequence comprising an AChE variant at its 5′ end. Said variant sequence is substantially as denoted by any one of SEQ. ID. Nos.1, 2, 3, 4, 5, 6, 7, 8, 9 and 10 (see FIG. 1 and Table 3), as well as functional analogues and derivatives thereof.

The diversified regulation at the 5′ UTR level may reflect yet unexplained roles for the 5′ variants. For example, in the human fetus, hE1d mRNA (the corresponding cDNA is herein denoted by SEQ. ID. No.10) was expressed in the nervous system and thymus in a development-dependent manner. In the fetal brain, hE1d mRNA was expressed in migrating neurons in both cell bodies and neuritic processes, and the number of hE1d-positive neurons grew from around zero, at week 16, to about 50% of the neurons at week 34, coinciding with the formation of synapses in these neurons.

By “analogues and derivatives” is meant the “fragments”, “variants”, “analogs” or “derivatives” of said nucleic acid molecule. A “fragment” of a molecule, such as any of the cDNA sequences of the present invention, is meant to refer to any nucleotide subset of the molecule. A “variant” of such molecule is meant to refer a naturally occurring molecule substantially similar to either the entire molecule or a fragment thereof. An “analog” of a molecule can be without limitation a paralogous or orthologous molecule, e.g. a homologous molecule from the same species or from different species, respectively. Functional analogues and derivatives exert the same activities as the native molecule.

The term “within the degeneracy of the genetic code” used herein means possible usage of any nucleotide combinations as codons that code for the same amino acid. In other words, such changes in the nucleic acid sequence that are not reflected in the amino acid sequence of the encoded protein.

Specifically, an analogue or derivative of the nucleic acid sequence of the invention may comprise at least one mutation, point mutation, nonsense mutation, missense mutation, deletion, insertion or rearrangement.

The novel exons described herein, when translated, provide a peptide comprising AChE transmembrane and intracellular domains. Said peptide may be from mouse or human origin, and thus is denoted by SEQ. ID. No.11 (mouse) or SEQ. ID. Nos. 12, 13 and 14 (human) (see FIG. 6 and Table 3), as well as functional analogues and derivatives thereof.

The amino acid sequence of an analog or derivative may differ from said AChE transmembrane and/or intracellular domain of the present invention when at least one residue is deleted, inserted or substituted.

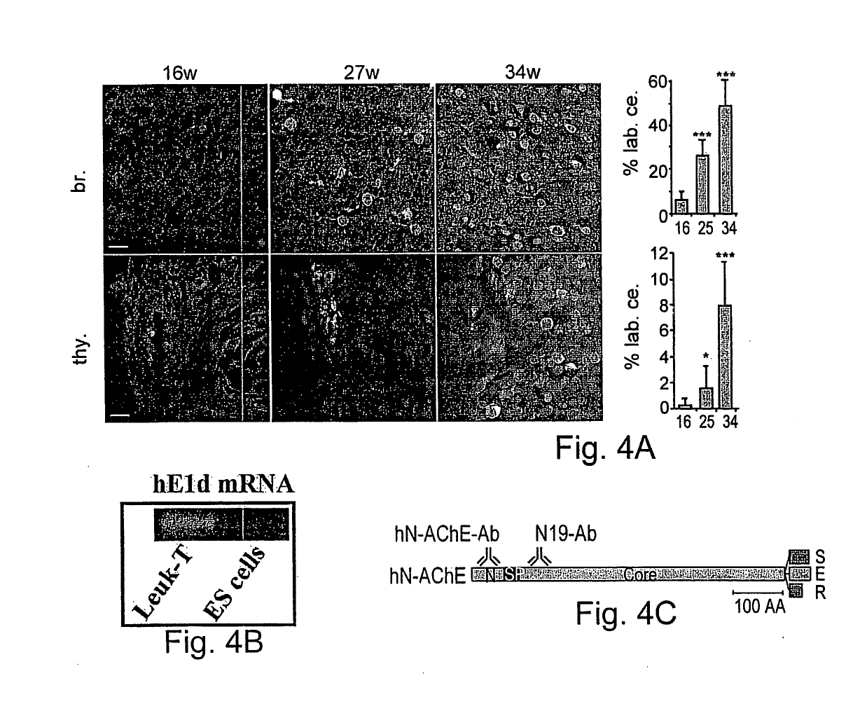

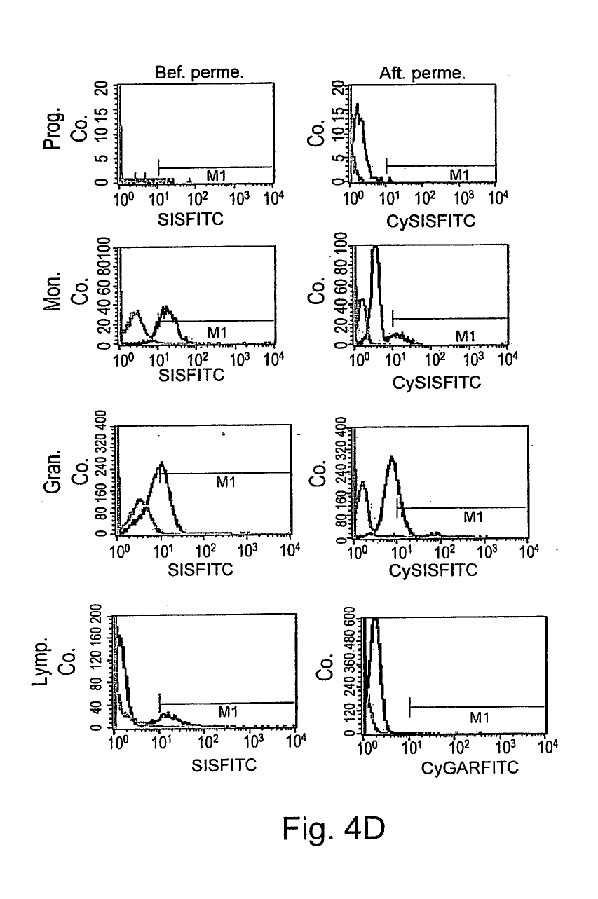

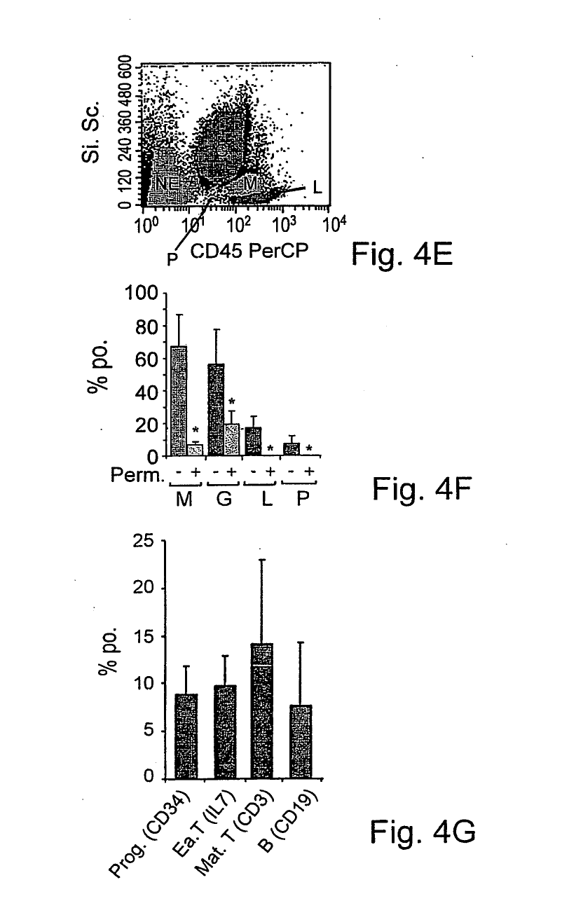

In addition, the present invention provides an AChE protein comprising a transmembrane domain. Thus, the novel AChE protein is comprised of an extracellular, a transmembrane and an intracellular domain, which may be of the -S, -R or -E forms, denoted by sequences SEQ. ID. Nos.15, 16 and 17 (see Table 3 and FIG. 4), respectively, as well as functional analogues or derivatives thereof.

However, it is to be understood that the invention pertains to any peptide comprising a sequence structurally similar to the novel transmembrane AChE domain, or a protein comprising a sequence structurally similar to the novel N-AChE sequence, with substantially equal or greater activity. Changes in the structure of the peptide or the protein comprise one or more deletions, additions, or substitutions. The number of deletions or additions, which may occur at any point in the sequence, including within the AChE-derived sequence, will generally be less than 25%, preferably less than 10% of the total amino acid number.

Preferred substitutions are changes that would not be expected to alter the secondary structure of the peptide, i.e., conservative changes. The following list shows amino acids that may be exchanged (left side) for the original amino acids (right side).

| Original Residue | Exemplary Substitution |
|---|---|
| Ala | Gly; Ser |
| Arg | Lys |
| Asn | Gln; His |
| Asp | Glu |
| Cys | Ser |
| Gln | Asn |
| Glu | Asp |
| Gly | Ala; Pro |
| His | Asn; Gln |
| Ile | Leu; Val |
| Leu | Ile; Val |
| Lys | Arg; Gln; Glu |
| Met | Leu; Tyr; Ile |
| Phe | Met; Leu; Tyr |
| Ser | Thr |
| Thr | Ser |
| Trp | Tyr |
| Tyr | Trp; Phe |
| Val | Ile; Leu |

Amino acids can also be grouped according to their essential features, such as charge, size of the side chain, and the like. The following list shows groups of similar amino acids. Preferred substitutions would exchange an amino acid present in one group with an amino acid from the same group.
1. Small aliphatic, nonpolar: Ala, Ser, Thr Pro, Gly;
2. Polar negatively charged residues and their amides: Asp, Asn, Glu, Gln;
3. Polar positively charged residues: His, Arg, Lys;
4. Large aliphatic nonpolar residues: Met, Leu, Ile, Val, Cys;
5. Large aromatic residues: Phe, Tyr, Trp.

Further comments on amino acid substitutions and protein structure may be found in Schulz et al., Principles of Protein Structure, Springer-Verlag, New York, N.Y., 1798, and Creighton, T. E., Proteins: Structure and Molecular Properties, W.H. Freeman & Co., San Francisco, Calif. 1983.

The preferred conservative amino acid substitutions as detailed above are expected to substantially maintain or increase the function or activity of the peptide or protein of the invention, as detailed hereinbelow. Of course, any amino acid substitutions, additions, or deletions are considered to be within the scope of the invention where the resulting peptide or protein is a peptide or protein of the invention which is substantially equal or superior in terms of function. In one specific example, the amino acid substitution(s), addition(s), or deletion(s) may be such that the resulting AChE protein is soluble or secreted when produced in a protein expression system.

The peptides and the protein provided by the invention may be isolated, synthetic or recombinantly produced.

In another aspect, the present invention provides a nucleic acid construct comprising any one of the sequences denoted by SEQ. ID. Nos.1-10 and 36-38, operably linked to at least one control element.

In one embodiment said construct may be an expression vector.

“Expression Vectors”, as used herein, encompass plasmids, viruses, bacteriophages, integratable DNA fragments, and other vehicles, which enable the integration of DNA fragments into the genome Of the host. Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. Generally, the genetic control elements can include a prokaryotic promoter system or a eukaryotic promoter expression control system. Such system typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

The term “operably linked” is used herein for indicating that a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame. Thus, a DNA sequence and a regulatory sequence(s) are connected in such a way as to permit gene expression when the appropriate molecules (e.g., transcriptional activator proteins) are bound to the regulatory sequence(s). The recombinant nucleic acid molecule to be introduced into the host cell may optionally further comprise an operably linked terminator which is functional in the host cell of choice. The recombinant nucleic acid molecule of the invention may optionally further comprise additional control, promoting and regulatory elements and/or selectable markers, which are operably linked to the recombinant nucleic acid molecule.

A vector may additionally include appropriate restriction sites, antibiotic resistance or other markers for selection of vector containing cells. Plasmids are the most commonly used form of vector but other forms of vectors which serves an equivalent function and which are, or become, known in the art are suitable for use herein. See, e.g., Pouwels et al. Cloning Vectors: a Laboratory Manual (1985 and supplements), Elsevier, N.Y.; and Rodriguez, et al. (eds.) Vectors: a Survey of Molecular Cloning Vectors and their Uses, Buttersworth, Boston, Mass. (1988), which are fully incorporated herein by reference.

In general, such vectors contain in addition specific genes, which are capable of providing phenotypic selection in transformed cells. The use of prokaryotic and eukaryotic viral expression vectors to express the genes coding for the polypeptides of the present invention are also contemplated.

The vector is introduced into a host cell by methods known to those of skilled in the art. Introduction of the vector into the host cell can be accomplished by any method that introduces the construct into the cell, including, for example, calcium phosphate precipitation, microinjection, electroporation or transformation. See, e.g., Current Protocols in Molecular Biology, Ausubel, F. M., ed., John Wiley & Sons, N.Y. (1989).

In a further aspect, the present invention provides a transfected cell containing an exogenous sequence, wherein said cell is transfected with the construct of the invention, or with any one of the sequences corresponding to the novel 5′ AChE variants described herein.

“Cells”, “host cells” or “recombinant host cells” are terms used interchangeably herein. It is understood that such terms refer not only to the particular subject cells but to the progeny or potential progeny of such a cell. Because certain modifications may occur in succeeding generation due to either mutation or environmental influences, such progeny may not, in fact, be identical to the parent cell, but are still included within the scope of the term as used herein. “Host cell” as used herein refers to cells which can be recombinantly transformed with naked DNA or expression vectors constructed using recombinant DNA techniques. As used herein, the term “transfection” means the introduction of a nucleic acid, e.g., naked DNA or an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of the desired protein.

A variety of cells is suitable for transfection, and may be selected according to the protein expression system of choice, which include bacteria (including Gram-negative and Gram-positive organisms, e.g., E. coli and B. subtilis), yeast (e.g. S. cerevisiae and Pichia), other lower eukaryotes like species of the genus Dictyostelium, tissue culture cell lines from animal cells, both of non-mammalian origin, e.g., insect and bird cells, as well as of mammalian origin, e.g., human and other primate cells, rodent cells, and plant cells.

Thus, the expression vector comprising the nucleic acid sequence encoding the AChE protein of the invention must, besides having all the required elements as described above, be in accordance with the protein expression system of choice. Usually, expression vectors are available commercially.

Sequences encoding signal peptides may be joined to sequences encoding the proteins of the present invention. The use of a signal sequence may be advantageous for expression of recombinant proteins in either prokaryotic or eukaryotic hosts. Secretion signals are relatively short (16-40 amino acids) in most species. The presence of a signal sequence on the protein permits the transport of the protein into the periplasm (prokaryotic hosts) or the secretion of the protein (eukaryotic hosts). Signal sequences from bacterial or eukaryotic genes are highly conserved in terms of function, although not in terms of sequence, and many of these sequences have been shown to be interchangeable [Grey, G. L. et al. (1985) Gene 39:247]. The presence of a signal sequence, on a protein expressed in a eukaryotic host cell, results in the transport of the nascent protein across the lumen of the rough endoplasmic reticulum, which may allow for eventual secretion of the protein into the culture medium. In both prokaryotes and eukaryotes, the signal sequence is removed from the amino-terminus of the protein molecule by enzymatic cleavage during transport of the polypeptide through the membrane.

Animal and plant protein expression systems differ greatly in protein glycosylation sequences, caused by differences in biosynthetic pathways. Protein glycosylation involves essentially the addition of a carbohydrate moiety, and it is one of the most common post-translational modifications of proteins. There are a few types of glycosylations, including the N-linked, which is more abundant, and where the glycan moiety is attached through Asn residues, and the O-linked, which are relatively scarce, where the glycan moiety is attached through Ser or Thr residues, as well as C-mannosylation, phosphoglycation, and glypiation. A comprehensive review on protein glycosylation may be found in Spiro, R. G. (2002) Glycobiology 12 (4): 43R-56R. When the host cell is a plant cell, it may be, e.g., a plant root, a celery, a ginger, a horseradish or a carrot cell. Any of these cells may be transformed with, e.g., Agrobacterium rhizogenes. Accordingly, regulatory elements that may be used in the expression constructs adapted to plant cells include promoters which may be either heterologous or homologous to the plant cell. The promoter may be a plant promoter or a non-plant promoter which is capable of driving high levels transcription of a linked sequence in plant cells and plants. The expression vectors used for transfecting or transforming the host cells of the invention can be additionally modified according to methods known to those skilled in the art to enhance or optimize heterologous gene expression in plants and plant cells. Such modifications include but are not limited to mutating DNA regulatory elements to increase promoter strength or to alter the AChE protein of the invention. Protein production from plant cells has been described in WO 04/096978.

Thus, the present invention also contemplates a method for the production of recombinant AChE protein, making use of a specific protein expression system and matching expression vector, as exemplified above. Said recombinant AChE protein produced is any AChE variant having AChE activity. Known AChE variants are N-AChE-R, N-AChE-E, as described herein, as well as AChE-R [Genbank Accession No. DQ140347; U.S. Pat. No. 6,025,183], AChE-S [U.S. Pat. No. 5,595,903] and AChE-E.

Furthermore, the recombinantly produced AChE protein may be further purified through any protein purification system known in the literature, like affinity or immuno-affinity chromatography, protein precipitation, e.g. ammonium sulfate fractionation, buffer exchange, ionic exchange chromatography, hydrophobic exchange chromatography and size-exclusion chromatography. Purification is usually followed by electrophoresis analysis and assaying for specific activity. Further, the purified protein may be concentrated by lyophilization or ultrafiltration.

It has been previously described that in brain neurons, AChE mRNA is subject to stress-related regulation and neuritic translocation, stress-responding neurons display replacement of dendritic AChE-S with ACNE-R mRNA [Meshorer 2002 id ibid.]. Alternative first exons could possibly influence the cellular and subcellular distribution of the different transcripts. It remains to be tested which and if the newly identified first exons could be regulated in a similar manner. None of the three 5′ murine probes tested (mE1a, mE1b and mE1d) showed dendritic expression in control mice, but in murine Purkinje cells, mE1d presented an unusual subcellular expression in both cell bodies and axons. This observation sets novel questions regarding the expression pattern and the physiological function of AChE in Purkinje cells in general, and in axonal processes in particular.

Hence, the present invention provides a marker for one of stress, cholinergic balance, and Alzheimer's disease, wherein said marker consists of an AChE mRNA comprising a variant 5′ region (essentially as denoted by any one of SEQ. ID. Nos. 3, 4 and 5, see Table 3). The glucocorticoid and stress dependence of the new exons suggests the use of such markers to identify hormone and stress-induced diseases.

Said marker may not be responsive to cortisol treatment, in which case said variant 5′ region is essentially as denoted by SEQ. ID. No. 3, as well as functional analogues and derivatives thereof.

When said marker is responsive to cortisol treatment, and said variant 5′ region is essentially as denoted by any one of SEQ. ID. Nos: 4 and 5, as well as functional analogues and derivatives thereof.

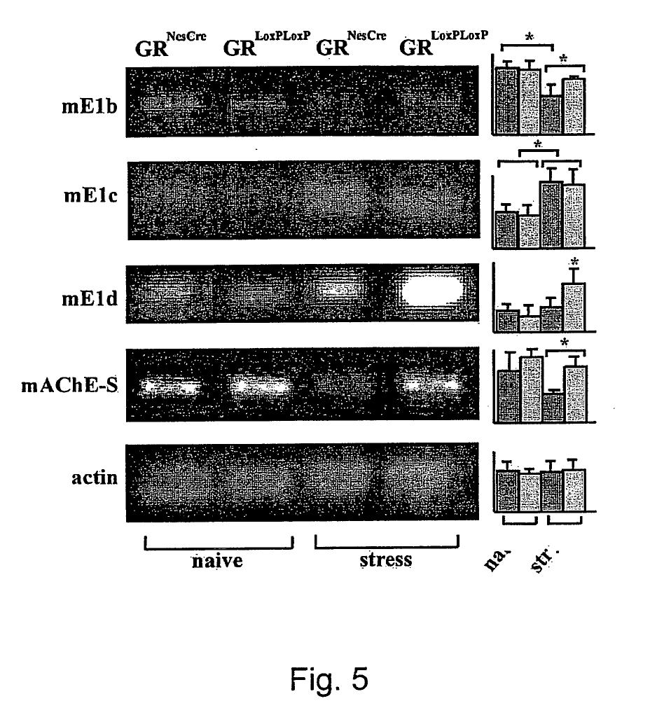

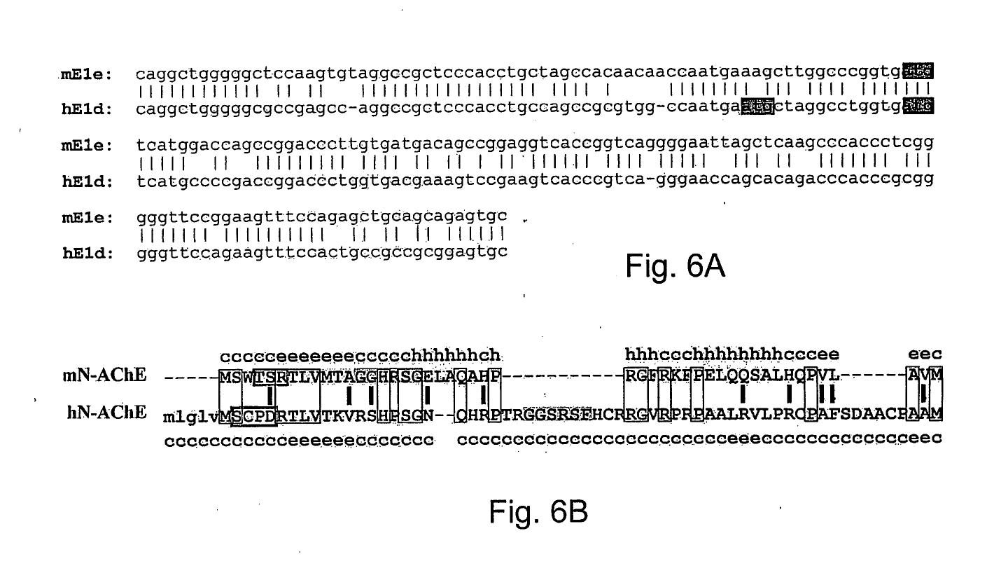

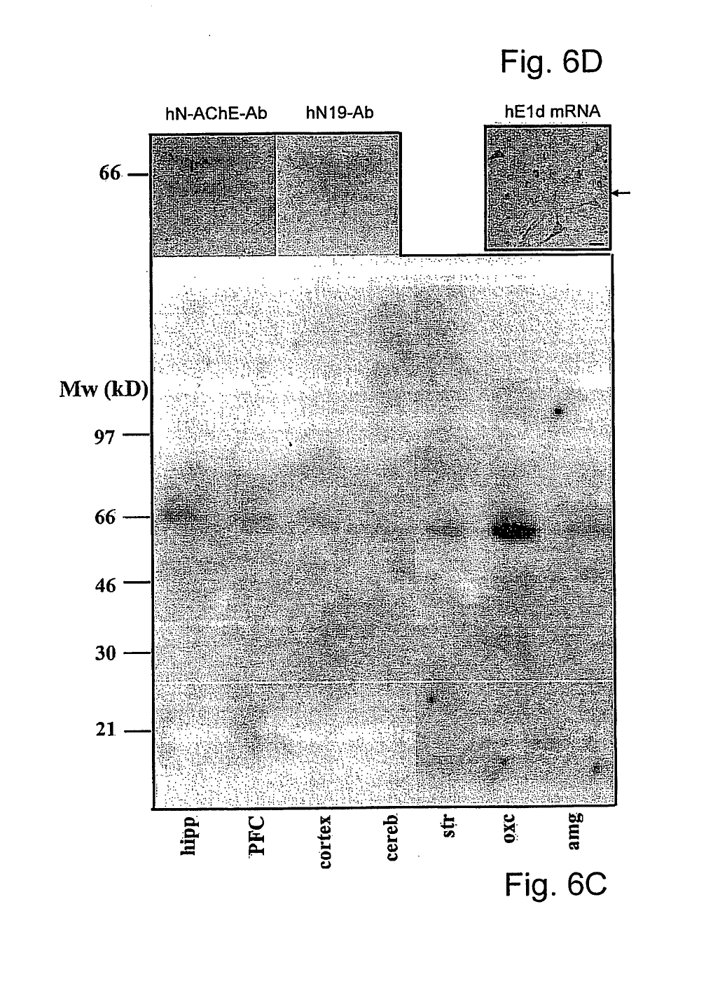

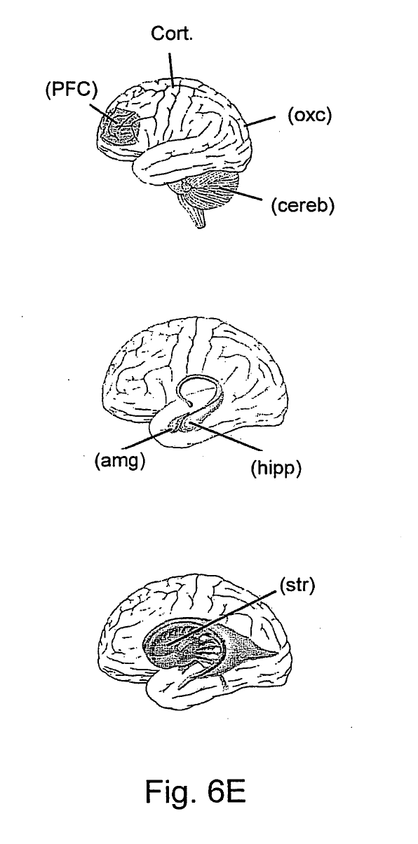

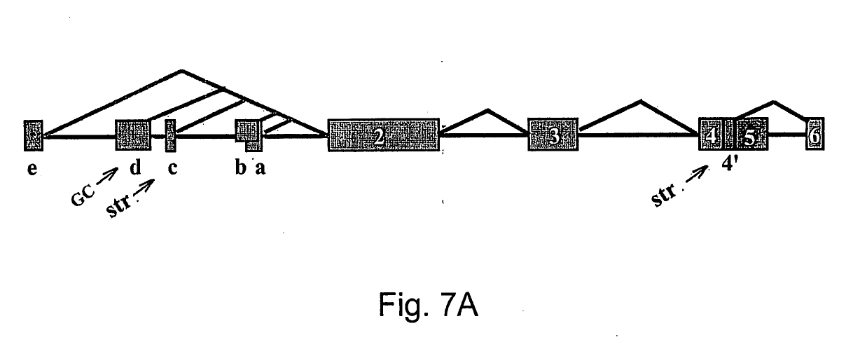

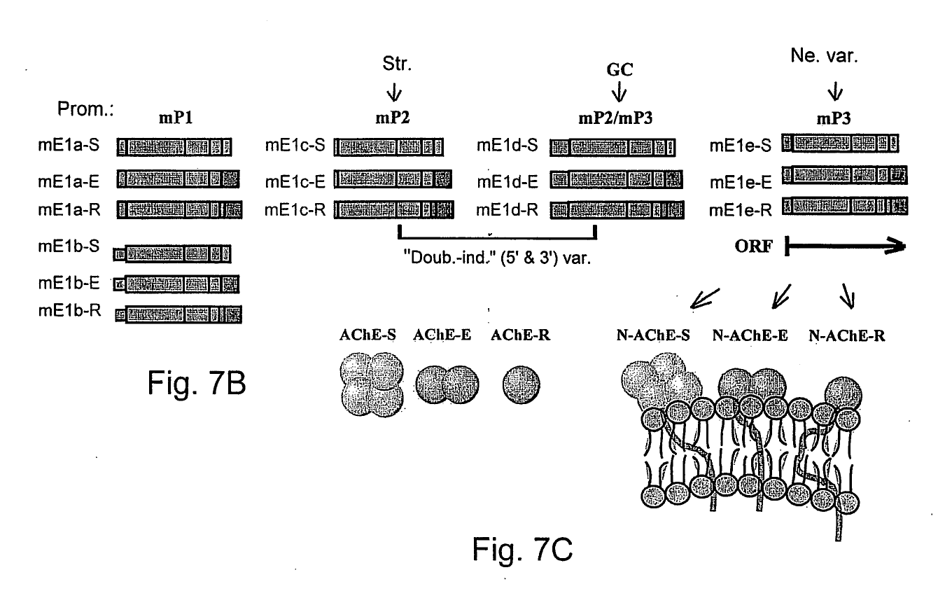

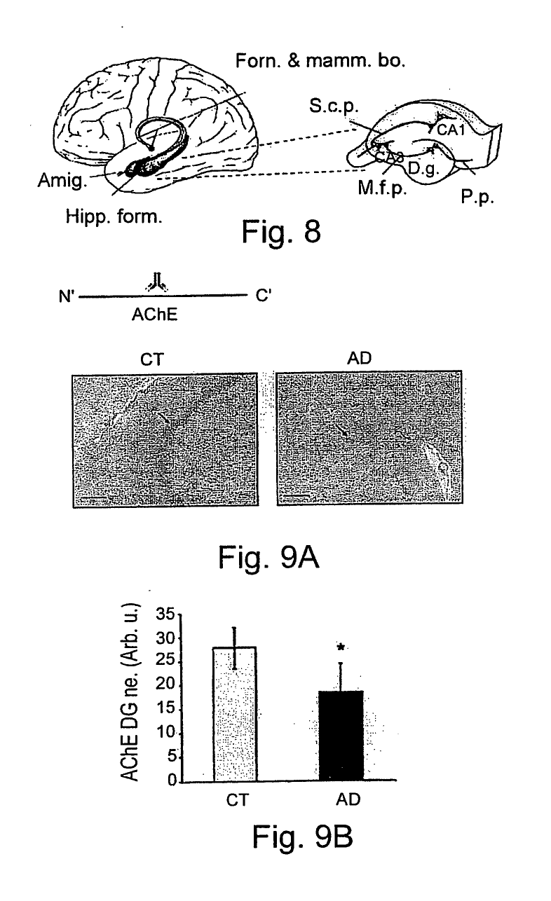

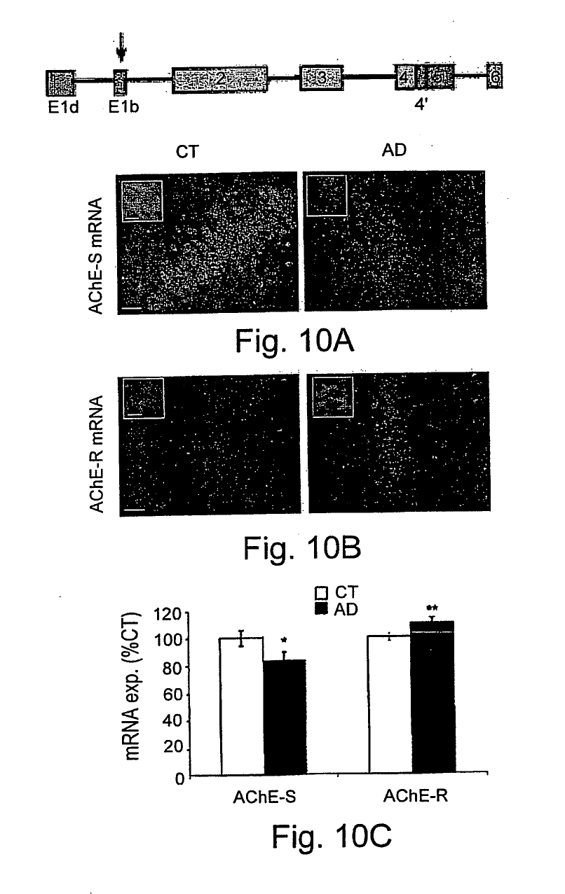

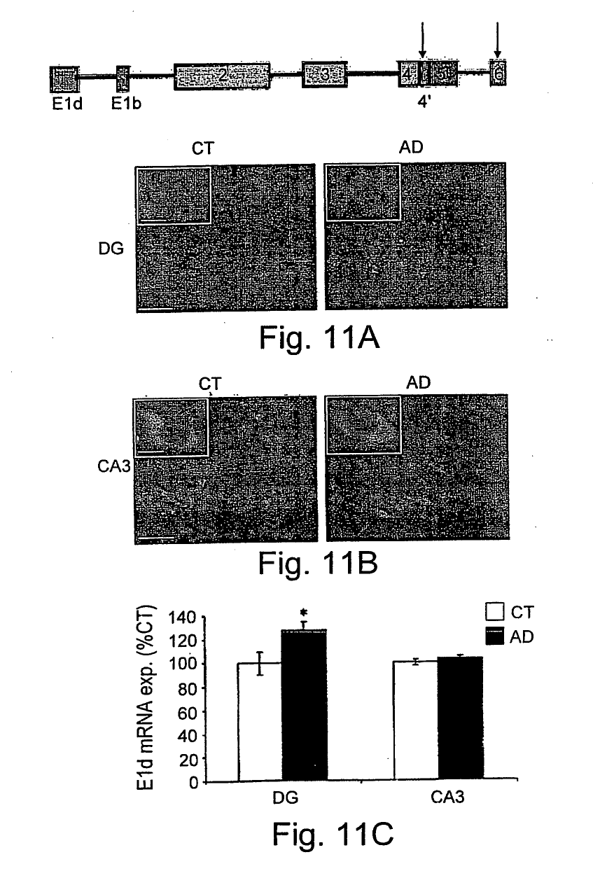

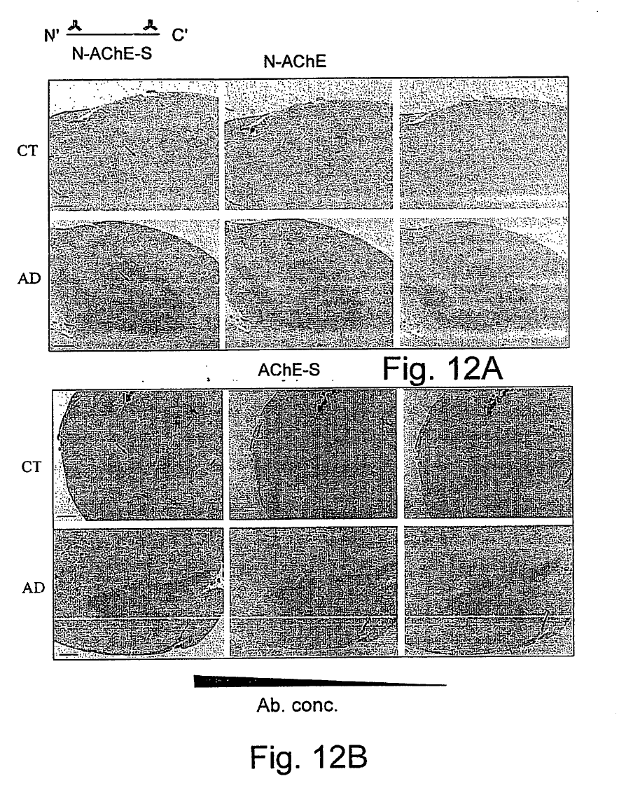

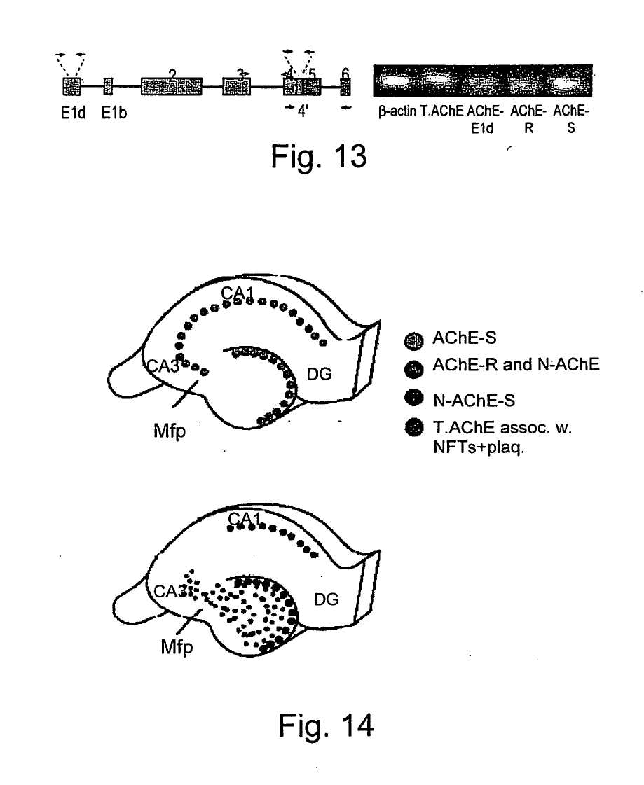

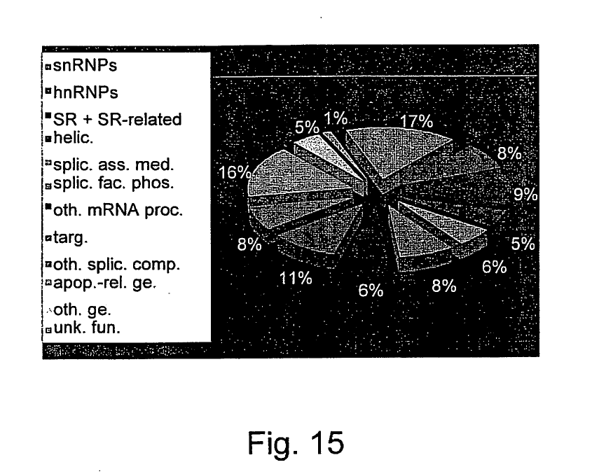

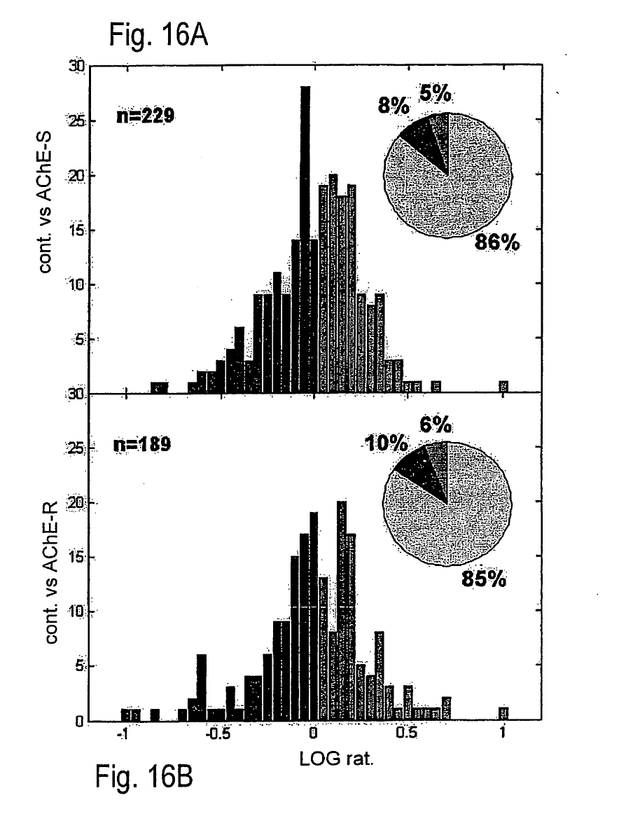

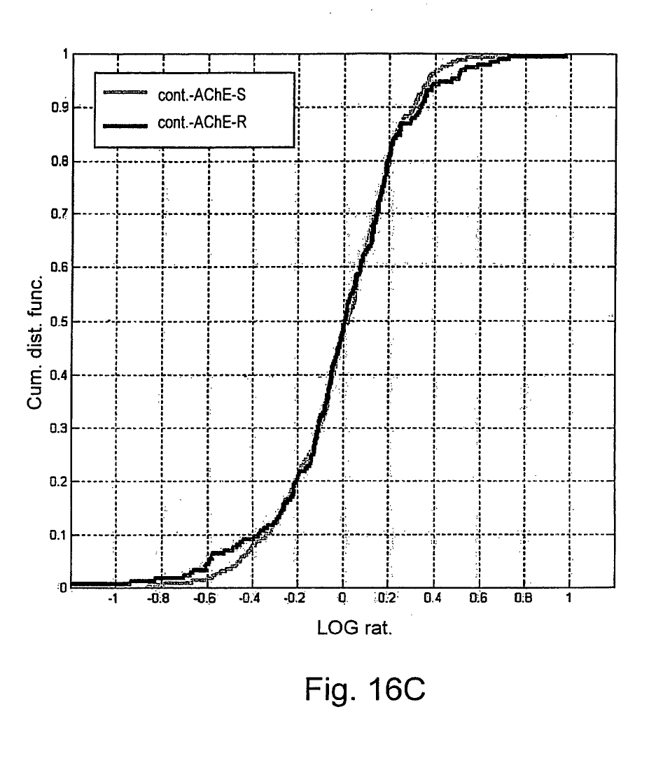

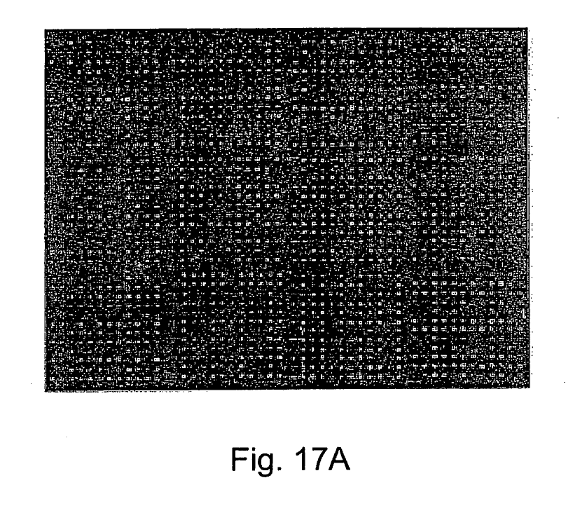

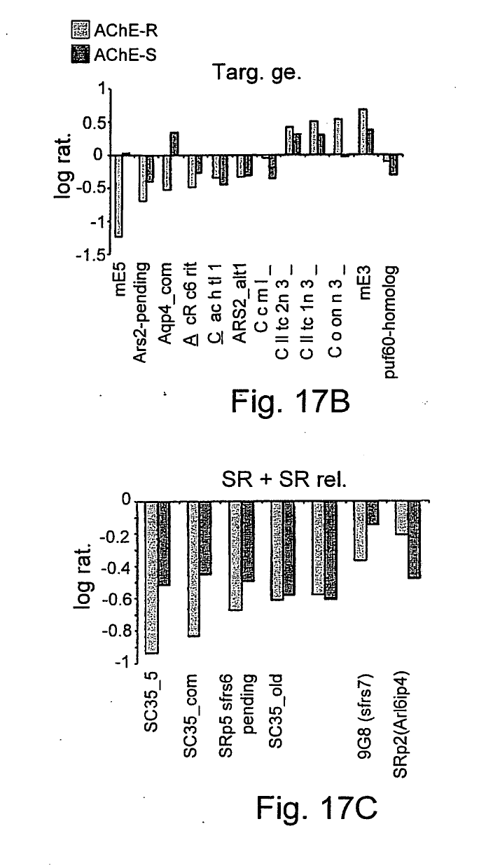

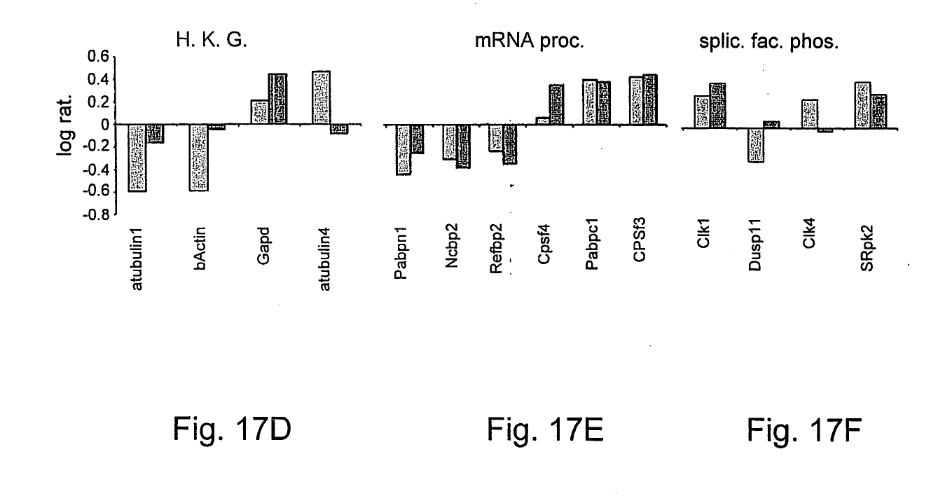

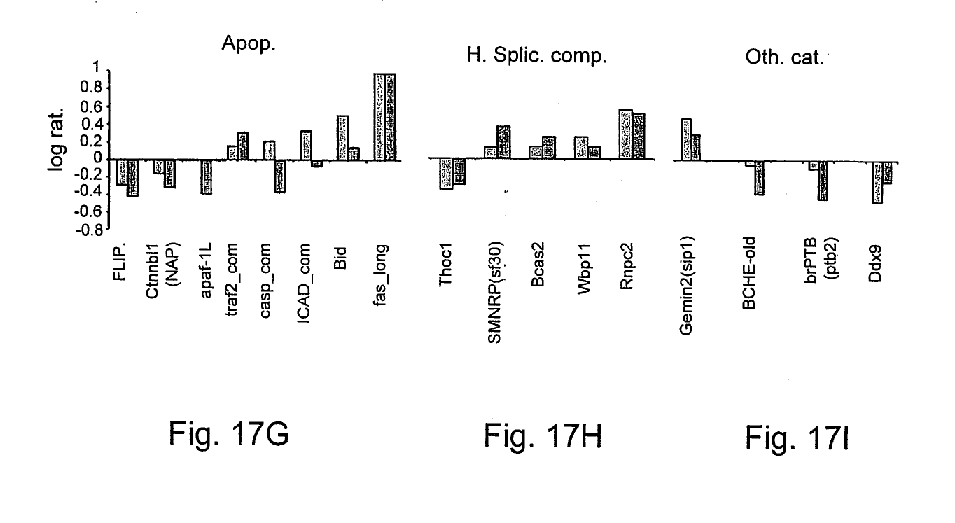

In the present study, the inventors explored whether the newly described transcripts are differentially regulated under stress and, if so, whether stress-induced release of glucocorticoids (GCs) is involved. A GRE site was identified inside exon mE1d, AP1 sites were found in mP2 as well as mP3, and two GREs are located upstream to mE1b. The distribution of glucocorticoid-responsive and stress-responsive elements thus predicted distinct responses of the various new exons. Therefore, the inventors studied their expression in control and in glucocorticoid receptor (GR) mutant mice deprived of neuronal GR [Tronche, F. et al. (1999) Nat Genet. 23, 99-103]. Two variants, mE1c and mE1d were found to be induced in response to immobilization stress. Of these two, only mE1d required the activation of GR for its induction (FIG. 5). In contrast, mE1b was repressed under stress, but only in GRNesCre mice, where GR does not bind to glucocorticoid response elements (GREs). This response is similar to that of AChE-S (FIG. 5B). One possible explanation could be that following stress, contrasting effects of different factors—among them GC—cancel out one another, keeping the levels of mE1b unaltered. However, in the absence of GR, the GREs are no longer functional. Maintained activities of suppressing factors may then reduce mE1b levels.

The novel 5′ alternative splicing patterns of AChE pre-mRNA are significant at several levels. First and foremost, they extend the complexity and versatility of AChE mRNA variants to levels that were not previously perceived. In addition, this study unveiled the existence of N-terminally extended membranal variant(s) of AChE (N-AChE) in brain neurons and hematopoietic cells. While the C-terminal composition and membranal directionality of these variants await further research, this finding explains certain long-known enigmas in AChE research and opens numerous new questions. The apparent conservation of this extended domain in rodents and primates strengthens the notion of its importance, and its unique expression patterns and stress-associated regulation call for exploring its functional significance.

The N-terminal amino acids of N-AChE (corresponding to the sequence MLGLVMSC, SEQ. ID. No.39) show the properties of a short signal peptide, suggesting that this protein may be secreted as well.

Having characterized new isoforms of AChE, the inventors generated an antibody, using as antigen two synthetic peptides (denoted by SEQ. ID. Nos 13 and 14), derived from the sequence encoded by the novel 5′ region. This antibody was able to identify the expression of the novel N-terminally extended AChE in tissues (FIG. 6C, FIG. 9A-9B, FIG. 12A-12B).

Thus, in a further aspect, the present invention provides an antibody recognizing an N-terminal AChE intracellular domain. Said antibody is directed against a synthetic peptide essentially as denoted by any one of SEQ. ID. Nos.13 and 14 (see Table 3 and FIG. 4), as well as any variants, fragments or derivatives thereof.

The antibody of the invention may be either monoclonal or polyclonal. It may be prepared against a synthetic peptide, such as e.g. SEQ. ID. No.13 or SEQ. ID. No.14, or prepared recombinantly by cloning techniques using any of the expression vectors of the invention, or a naturally occurring AChE variant comprising the transmembrane domain can be isolated and used as the immunogen. The polypeptides of the invention can be used to produce antibodies by standard antibody production techniques, well known to those skilled in the art. For example, as described generally by Harlow and Lane [Harlow and Lane (1988) Antibodies: a Laboratory Manual, Cold Spring Harbor Laboratory Press, Cold Spring Harbor, N.Y.].

For producing polyclonal antibodies a host, such as a rabbit or goat, is immunized with the protein or polypeptide, generally with adjuvant and, if necessary coupled to a carrier. Antibodies are collected from the sera of the hosts. The generation of polyclonal antibodies against proteins is described in Chapter 2 of Current Protocols in Immunology, Wiley and Sons Inc

For producing monoclonal antibodies, generally a mouse is immunized with the polypeptide or peptide fragment, and then splenic antibody producing cells are isolated. These cells are fused to provide hybridomas that secrete the required antibody. The antibodies are collected from the ascitis fluid of the host or from the tissue culture media of said hybridomas. The technique of generating monoclonal antibodies is described in many articles and textbooks, such as the above-noted Chapter 2 of Current Protocols in Immunology.

Fab and F(ab′)₂ and other fragments of the anti-N-AChE antibodies, which are typically produced by proteolytic cleavage, using enzymes such as papain (to produce Fab fragments) or pepsin (to produce F(ab′)₂ fragments), are also provided by the present invention.

For clinical applications, as described below, the anti-N-AChE antibodies of the invention may be improved through a humanization process, to overcome the human antibody to mouse (or rabbit, or rat) antibody response. Rapid new strategies have been developed recently for antibody humanization which may be applied for such antibody. These technologies maintain the affinity, and retain the antigen and epitope specificity of the original antibody [Rader, C. et al. (1998) Proc. Natl. Acad. Sci. USA, 95: 8910-8915; Mateo, C. et al. (1997) Immunotechnology 3: 71-81]. Unlike, for example, animal derived antibodies, “humanized” antibodies often do not undergo an undesirable reaction with the immune system of the subject.

Thus, as used herein, the term “humanized” and its derivatives refers to an antibody which includes any percent above zero and up to 100% of human antibody material, in an amount and composition sufficient to render such an antibody less likely to be immunogenic when administered to a human being. It is being understood that the term “humanized” reads also on human derived antibodies or on antibodies derived from non human cells genetically engineered to include functional parts of the human immune system coding genes, which therefore produce antibodies which are fully human.

In addition, the antibodies of the invention can be bound to a solid support substrate and/or conjugated with a detectable moiety, as is well known in the art. The detectable moieties contemplated within the present invention can include, but are not limited to, fluorescent, luminescent, metallic, enzymatic and radioactive markers such as biotin, gold, ferritin, alkaline phosphatase, peroxidase, fluorescein, rhodamine, tritium, ¹⁴C and iodine.

The antibodies of the invention are also provided in the form of a composition. The preparation of pharmaceutical compositions is well known in the art and has been described in many articles and textbooks, see e.g., Remington's Pharmaceutical Sciences, Gennaro A. R. ed., Mack Publishing Co., Easton, Pa., 1990, and especially pp. 1521-1712 therein.

Further, the present invention provides the use of anti-AChEs, as well as the above-described antibody for intracellular signaling in cells expressing the AChE transmembrane domain (denoted by SEQ. ID. No.34). Said antibody, and inhibitors, may also be used as a ligand for AChE. Therefore, cells expressing this variant may serve as extremely sensitive biosensors, which would respond to binding of inhibitors or antibodies, by modifying intracellular signaling, through the kinase binding domain of N-AChE.

Antibodies generated against the hN-AChE peptide interacted with brain-expressed protein(s) with similar electrophoretic properties to those of AChE (FIG. 6). In addition, some of the commercially-available anti-AChE antibodies yield double bands around 66-70 kDa [see, for example Brenner et al. (2003) FASEB J. 17(2): 214-22]. This supports the notion that at least part of the brain AChE protein as known is N-terminally extended.

Another aspect provided by the present invention is a sensor for a cholinergic signal, wherein said sensor comprises the AChE extracellular, transmembrane and intracellular domains, denoted by any one of SEQ. ID. Nos. 11 and 12 (Table 3).

The N-terminus of hN-AChE likely thus enables monomeric AChE-S or AChE-R to transverse through the membrane, conferring yet undefined physiological functions by its cytoplasmic domain. Direct docking of AChE to the synaptic membrane would explain its presence in brain regions lacking the PRiMA subunit necessary to anchor AChE-S tetramers to the synapse [Perrier et al. (2003) Eur. J. Neurosci. 18(7): 1837-47]. This could have especially significant outcome for post-stress situations, where large amounts of monomeric AChE are produced rapidly. Membrane targeting of the produced enzyme could be cost-efficient for rapidly reducing the synaptic levels of ACh, whereas its putative N-terminal phosphorylation and farnesylation can possibly transduce cytoplasmic signals.

In a different aspect, the sensor of stress and cholinergic imbalance may be provided by the use of a cell expressing a AChE transmembrane domain, wherein said transmembrane domain is as described above.

In a yet further aspect, the present invention also provides a plurality of sensors for cholinergic signaling, embedded in (or affixed to) a suitable solid matrix. These sensors, when blocked with organophosphates or any anti-cholinesterases, will send a signal which would activate the kinase binding domain in the intracellular region of N-AChE and induce a signal transduction cascade which would be selective for this N-AChE variant alone.

Flow cytometry analyses demonstrated that hN-AChE is primarily located in blood cell membranes. Monocytes, granulocytes, lymphocytes, and CD34+ progenitors were all positive, albeit to different extents. In lymphocytes, hN-AChE levels increased from early to mature T-lymphocytes, possibly explaining the distinct expression patterns throughout thymic development. hN-AChE expression in T and B lymphocytes are compatible with reports of cholinergic regulation of lymphocytic functioning [Kawashima and Fujii (2000) Pharmacol. Ther. 86: 29-48].

The fact that the novel variants were detected in different lymphoid lineages at specific stages of development, as shown in FIG. 4C, suggested that these novel variants may be a marker for lymphoid cell lineage differentiation, wherein said marker comprises the sequence substantially as denoted by any one of SEQ. ID. Nos.11 and 12 (see Table 3), as well as any fragments, derivatives and analogues thereof, and wherein a decrease in the level of its expression denotes a more advanced stage of lymphoid differentiation.

Another finding related to the novel AChE isoform described herein (the N-AChE) refers to its correlation with Alzheimer's Disease. Impaired cholinergic neurotransmission is the major hallmark of Alzheimer's disease. However, the molecular mechanisms underlying this feature are not yet known. In Example 11, the inventors report increases of the extended 5′ variant of acetylcholinesterase (AChE) mRNA in hippocampal dentate gyrus (DG), but not CA3 neurons of Alzheimer's disease patients, as compared to non-demented controls (p<0.01, Student's t test) (FIGS. 10A-10C and 11A-11C). Antibodies directed at N-AChE revealed accumulation of the N-AChE variant at the mossy fiber system connecting the dentate gyrus to the CA3 region (FIG. 12A). Parallel accumulation was observed of the synaptic AChE variant, AChE-S (FIG. 12B), suggesting that Alzheimer's disease brains overexpress an N-terminally extended N-AChE-S protein in the dentate gyrus but not in CA3 neurons. A parallel decrease in ‘synaptic’ AChE (AChE-S, p<0.01) and an increase in ‘readthrough’ AChE (AChE-R, p<0.05) mRNA levels suggests that much of the AChE-S protein had been replaced by N-AChE-S and/or N-AChE-R. The unique biochemical composition of the N-terminal extension, combined with the membrane-adherent capacity of the AChE-S C-terminus, call for exploring the physiological consequences of N-AChE-S accumulation in the Alzheimer's disease hippocampus.

Thus, neuronal accumulation of the N-AChE isoform may be causally involved in Alzheimer's disease, and thus serve a diagnostic purpose. The anti-N-AChE antibodies may be used as a diagnostic tool, or, alternatively, for the therapeutics which would spare the normal enzyme while shutting the N-AChE down.

Positron Emission Tomography (PET), as well as Single Photon Emission Computerized Tomography (SPECT), are techniques that have been used in brain imaging [Kilbourne et al. (1996) Synapse 22: 123]. Both techniques can monitor non-invasively, using positron β+ or γ cameras, the time-course of regional tissue radioactive concentration ater administration of a compound labeled with a β+ or γ photon emitting radionuclide, respectively.

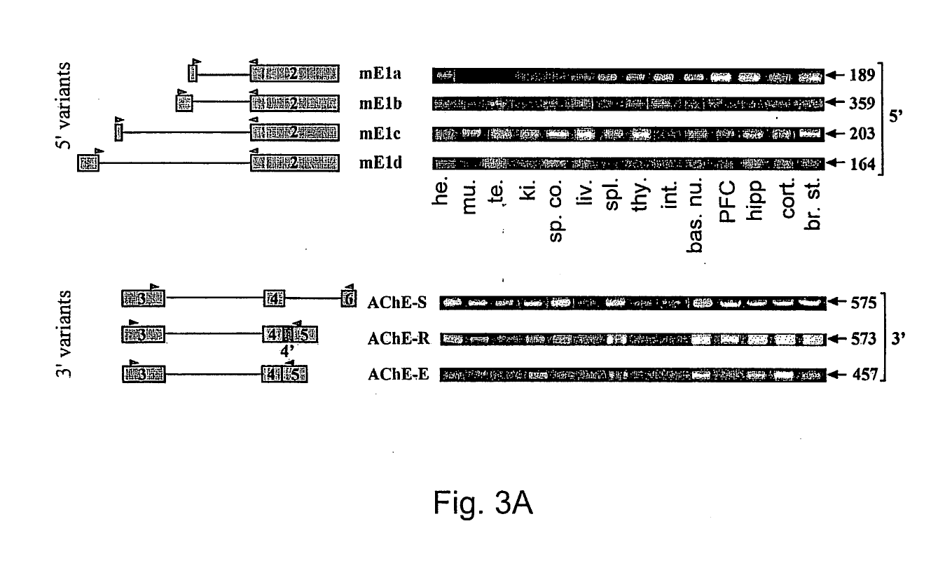

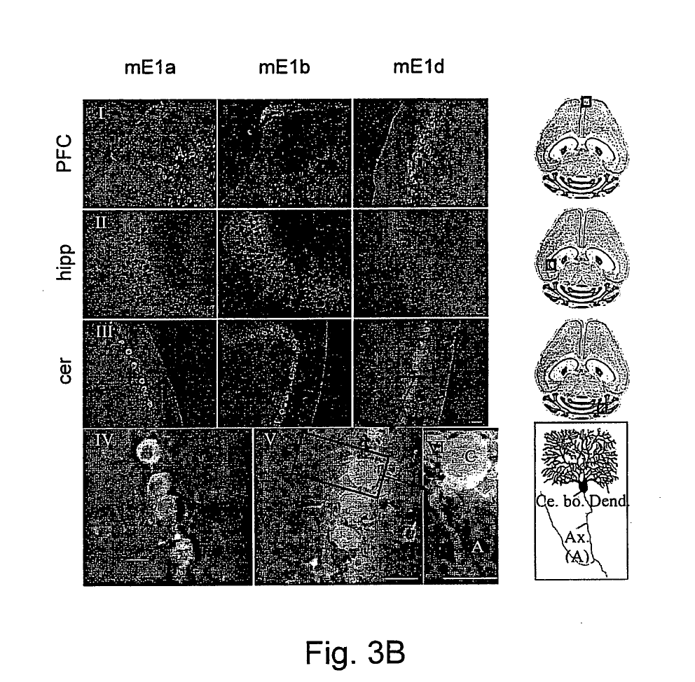

To date, Alzheimer's Disease can be diagnosed with certainty, but N-AChE is also expressed in the normally aged brain (FIG. 3A-3B), where it may trigger neuronal processes facilitating the disease process. Thus, the present invention presents a method of diagnostic, whereby the anti-N-AChE antibody of the invention is labeled with a radiotracer (a detectable marker), and administered to a subject in need. The subject then undergoes a PET or a SPECT scan, and binding of the antibody to the N-AChE of the hippocampus shall provide the evidence of Alzheimer's disease. This method is safe and non-invasive, because of blood brain barrier disruption in Alzheimer's disease, and the radioisotopes used have a short half-life, thus being weakly irradiating. Moreover, the diagnostic tool (the antibody) is known to interact selectively and specifically with its target, the N-AChE isoform, an excess of which has been correlated with Alzheimer's disease (as described in Example 11 below). This method provides an image of the human brain which shows the location and relative amount of N-AChE.

For PET scan, the main positron emitter radionuclides used for labeling the antibody are Carbon 11 [¹¹C], having a 20.4 min half-life, Fluorine 18 [¹⁸F], with a 110 min half-life, and Bromine 76 [⁷⁶Br], with a 16 hr half-life. All of these radionuclides need to be prepared with very high specific activity in a cyclotron. For SPECT scan, Iodine 123 [¹²³I], with a 31.2 hr half-life, may be used. This radioisotope is commercially available with very high specific activity.

A further inference from the inventors' present findings involves the correlation between the overexpression of N-AChE in Alzheimer's hippocampus, and the apoptotic fate of the basal nuclei neurons in this condition. Interestingly, the ACHE mRNA transcrips further undergo 3′ alternative splicing, as demonstrated herein and in the inventors' previous reports [Soreq and Seidman (2001) id ibid.].To find if these two phenomena are causally related, the inventors generated p19 cells overexpressing AChE-R or AChE-S and show, as described in Example 12, how overexpression of each of these two proteins affects the pattern of gene expression in these cells (which were already differentiated towards the neuronal lineage), altering the expression of genes related to the splicing machinery, apoptosis and helicases. Moreover, apoptosis is also a process that may be triggered by the alternative splicing of other genes, such as e.g. the Bcl-2 gene [Stamm et al. (2005) Gene. 344:1-20. Epub 2004 Dec. 10].

The present invention is defined by the claims, the contents of which are to be read as included within the disclosure of the specification.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following Examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the intended scope of the invention.

### EXAMPLES

### Experimental Procedures

General Methods of Molecular Biology: A number of methods of the molecular biology art are not detailed herein, as they are well known to the person of skill in the art. Such methods include PCR, expression of cDNAs, transfection of mammalian cells, protein expression protocols and the like. Textbooks describing such methods are, e.g., Sambrook et al. (1989) Molecular Cloning, A Laboratory Manual, Cold Spring Harbor Laboratory, ISBN: 0879693096; F. M. Ausubel (1988) Current Protocols in Molecular Biology, ISBN: 047150338X, John Wiley & Sons, Inc. Furthermore, a number of immunological techniques are not in each instance described herein in detail, like for example Western Blot, as they are well known to the person of skill in the art. See, e.g., Harlow and Lane (1988) Antibodies: a laboratory manual. Cold Spring Harbor Laboratory.

Human tissues: The use of human embryos, cord blood, and adult tissue in this study was approved by the Tel-Aviv Sourasky Medical Center Ethics Committee according to the regulations of the Helsinki accords. Human embryos were transferred immediately to 4% PFA, embedded in paraffin and sliced (7 μm). Fresh samples of umbilical CB cells were obtained following normal deliveries. Adult human brain samples were collected within 4 hrs post-mortem from a 70 year-old patient with cardiac arrhythmias. Tissue was frozen immediately in liquid nitrogen. Brain homogenates (in 0.1M phosphate buffer, 1% Triton X-100) were immuno-blotted using standard procedures.

Animals: Central nervous system specific GR mutants (GR^{NEsCre}), control littermates (GR^{loxP/loxP}) [Tronche (1999) id ibid.] and FVB/N male mice were kept under 12 hr dark/12 hr light diurnal schedule, with food ad libitum. Stress experiments included 30 min immobilization in 50 ml conical tubes. Mice were sacrificed by decapitation 2 hr after immobilization, brains were dissected on ice and frozen in liquid nitrogen or fixed in 4% paraformaldehyde (PFA) for 24 hr, embedded in paraffin, sliced to 5-7 μm sections and collected by adhesion to Superfrost®-Plus slides (Menzel-Glaser, Braunschweig, Germany). For all experiments, naïve age-matched males served as controls. These experiment were approved by the animal committees in the Hebrew University and College de France.

Computational Resources: The human (GenBank Accession No. AF002993) and mouse (AF312033) ACHE loci were analyzed by the National Center for Biotechnology Information (www.ncbi.nlm.nih.gov) for access to the GenBank, as well as to Blast, Entrez, Locus Link, Structure, Protein, and OMIM database resources. Expert Protein Analysis System at the Swiss Institute of Bioinformatics (http://www.expasy.ch/) was used for access to a variety of data manipulation programs and protein databases. The Baylor College of Medicine (BCM) Search Launcher (http://searchlauncher.bcm.tmc.edu) served for data manipulation and to derive display programs. The MatInspector program at Genomatix (genomatix.gsf.de) or the Cister software (http://zlab.bu.edu/˜mfrith/cister.shtml) were used to find transcription factor binding sites.

RNA extraction and cDNA preparation: Total RNA was extracted from animal and human tissues using the EZ-RNA total RNA isolation kit (Biological Industries, Beit Haemek, Israel) as instructed, diluted in diethyl pyrocarbonate (DEPC) treated water to a concentration of 100 ng/μl and stored at −70° C. until use. Human RNA from leukemic T lymphocytes, liver and testis was obtained from Ambion (Austin, Tex., USA). SuperScript Reverse Transcriptase (Life Technologies, Gibco BRL, Bethesda, Md.) served for reverse transcription with either poly-dT or random hexamers. Gene-specific primers (see below) were used for one-step RT-PCR (Qiagen, Hilden, Germany).

FISH (Fluorescence In Situ Hybridization): Paraffin-embedded sections (mouse horizontal whole brain sections, human whole embryos saggital sections and human adult PFC) were subjected to deparaffination with xylene (2×5 min washes), followed by decreasing ethanol washes (100, 75, 50 and 25%) and then a wash in PBS with 0.5% Tween-20 (PBT) and incubation with 10 mg/ml proteinase K (8 min, room temp). Hybridization in a humidified chamber involved 10 mg/ml probe (in 50% formamide, 5×SSC, 10 mg/ml tRNA, 10 mg/ml heparin, 90 min, 52° C.). Sections were then washed twice at 60° C. with 50% formamide, 5×SSC and 0.5% sodium dodecyl sulfate (SDS), twice in 50% formamide, 2×SSC at 60° C., twice in Tris-buffered saline+0.1% Tween-20 (TBST) at room temp, and blocked in 1% skim milk (Bio-Rad, Hercules, Calif., USA) for 30 min.

Biotin-labeled probes (Table 1) were detected by incubating sections with streptavidin-Cy3 conjugates (CyDye™, Amersham Pharmacia Biotech, Little Chalfont, UK) for 30 minutes, followed by three washes in TBST. Sections were mounted with IMMU-MOUNT (Shandon Inc, Pittsburgh, Pa., USA).

TABLE 1
FISH probes for the novel 5′ exons

| | | | | |
|---|---|---|---|---|
| | Accession | | | SEQ. |
| Probe | Number | Sequence (5′-3′) | Position | ID. |

| | | | | |
|---|---|---|---|---|
| mE1a | AY389982 | CUGGUGUCAGAACUCAAGCC | 76-115 | No. 18 |
| | | CCUAUUGCAUCCCCAUAUUG | | |
| mE1b | AY389981 | CUCCCCGCCCGAGCCUUGGU | 175-222 | No. 35 |
| | | GUGGGGGUAUCUGGAGAAUC | | |
| | | GUGAGCAU | | |
| mE1d | AY389980 | UGUGUGACAGACGGACCGCA | 248-295 | No. 19 |
| | | GCCUGCGGAGACACCAGACA | | |
| | | CCGUUCAC | | |
| hE1d | AY389977 | UCGUCACCAGGGUCCGGUCG | 227-271 | No. 20 |
| | | GGGCAUGACAUCACCAGGCC | | |
| | | UAGCA | | |

Polymerase chain reaction: PCR was used for detecting different transcripts in various tissues and to confirm sequences. PCR reaction mixture contained 2 units Taq DNA polymerase (Sigma, St. Louis, Mo.), deoxynucleotide mix (0.2 mM each) (Sigma), forward/reverse primers (0.5 μM each, Table 2 below) and 300 ng of template (cDNA or genomic DNA). Each of 35 cycles included denaturation (1 min, 95° C.), annealing (1 min, 60° C.) and elongation (72° C., 1 min).

TABLE 2
PCR Primers

| | Accession | | | |
|---|---|---|---|---|
| Exon/Gene | Number | | Position | SEQ. ID. |
| | | Forward Primer (5′ - 3′) | | |
| mE1a | AF312033 | AGCGGAGGGCATTGCAATA | 8552-8570 | No. 21 |
| mE1b | AF312033 | TTTGATCTCTTGGCTGGAGA | 8333-8354 | No. 22 |
| | | CG | | |
| mE1c | AF312033 | GGAACATTGGCCGCCTCCAG | 7547-7567 | No. 23 |
| | | C | | |
| mE1d | AF312033 | CAGGCTGCGGTCCGTCTGTC | 7277-7297 | No. 24 |
| | | A | | |
| hE1d | AF002993 | CCTGGTGACGAAAGTCCGA | 13275-13257 | No. 25 |
| mAChE-R | AF312033 | CCGGGTCTATGCCTACATCT | 11016-11040 | No. 26 |
| | | TTGAA | | |
| mAChE-S | AF312033 | CGGGTCTATGCCTACATC | 11017-11034 | No. 27 |
| mAChE-E | AF312033 | CCGGGTCTATGCCTACATCT | 11016-11040 | No. 28 |
| | | TTGAA | | |
| | | Reverse Primer (5′ - 3′) | | |
| mE1a | AF312033 | CCAGCAGCTGCGGGTCTTCC | 9379-9398 | No. 29 |
| mE1b | AF312033 | CCAGCAGCTGCGGGTCTTCC | 9379-9398 | No. 29 |
| mE1c | AF312033 | CCAGCAGCTGCGGGTCTTCC | 9379-9398 | No. 29 |
| mE1d | AF312033 | CCAGCAGCTGCGGGTCTTCC | 9379-9398 | No. 29 |
| hE1d | AF002993 | TCCTCCACCCAGGAGCCAGA | 10746-10726 | No. 30 |
| | | G | | |
| mAChE-R | AF312033 | AAGGAAGAAGAGGAGGGACA | 12787-12814 | No. 31 |
| | | GGGCTAAG | | |
| mAChE-S | AF312033 | GCTCGGTCGTATTATATCCC | 13578-13598 | No. 32 |
| | | A | | |
| mAChE-E | AF312033 | AAGGAAGAAGAGGAGGGACA | 12787-12814 | No. 31 |
| | | GGGCTAAG | | |

Antibodies: High affinity polyclonal rabbit IgG antibodies against the human hE1d-encoded N-terminal domain were tailor-made (Eurogentec, Seraing, Belgium). Two 16 amino acids long peptides from the coding sequence of human exon hE1d (hN-AChE) were synthesized, mixed and injected together into two rabbits. Additional boost injections were given 2, 4 and 8 weeks thereafter. Final bleeding was carried out after week 16. ELISA screening with the synthetic peptides served to identify successful antibody production. The synthetic peptides were further used for affinity purification of the antibodies. A dilution of 1:500 of the affinity-purified antiserum was used for Western blotting. The two synthetic peptides used in the immunization are denoted by the following sequences: KVRSHPSGNQHRPTRG (also known as peptide 437, SEQ. ID. No. 13), and GSRSFHCRRGVRPRPA (also known as peptide 438, SEQ. ID. No. 14).

Flow cytometry: Mononuclear fractions of cord blood cells were separated on Ficoll-Hypaque gradients 1.077 g/cm3 (Pharmacia, Uppsala, Sweden) as described (Grisaru et al., 2001). Cells were permeabilized and fixed for 7 minutes (Fix and Perm Kit; Caltag, Burlingame, Calif.) then stained with PerCP-conjugated anti-CD34 (Becton-Dickinson [BD], Oxford, UK) or the other noted antibodies. Isotype controls served to distinguish specific labeling. Rabbit anti-hN-AChE antibodies were detected on these cells using fluorescein isothiocyanate (FITC)-conjugated goat anti-rabbit Fab antibodies (Jackson Immunoresearch Labs, Inc., Westgrove, Pa., USA). Multiparameter flow cytometry was performed using a FACScalibur (BD) and CellQuest software (BD). hN-AChE expression was assessed in fresh CD34+ cells by analyzing 3000 gated events. Positively stained populations were defined using FITC, PE, and perCP isotype controls (BD).

### Preparation of Microarray Slides:

### 1. Oligonucleotide Selection

The microarray used in Example 12 is a small in-house constructed DNA oligonucleotides microarray, which was designed specifically to fit the present research interests. More precisely, it primarily contains two main categories of oligonucleotides: genes encoding spliceosomal components, and apoptosis-related genes undergoing alternative splicing.

The mouse homologs of the putative complete set of human genes encoding the spliceosome components [Zhou, Z. et al. (2002) Nature 419: 182-5] were identified using online databases [Stamm (2005) id ibid.], and oligonucleotides which correspond to these genes were selected. Some of these proteins were not previously known to be associated with the splicing machinery. The genes in this category include, among others, SR proteins, snRNPs, splicing factors phosphorylating proteins and spliceosomal assembly mediators.

Many of the genes involved in apoptosis undergo alternative splicing. In some cases, the resulting variants have opposite effects on cell fate (i.e. one is pro-apoptotic and the other is anti-apoptotic). The inventors thus, searched for such genes and included them in the microarray.

In addition to the above subgroups, several probes for genes not belonging to neither of the above categories were included in the microarray.

The different functional groups and their relative representation in the microarray are depicted in FIG. 15.

### Preparation of RNA Samples, Amplification, Labeling, Fragmentation, Pre-hybridization and Hybridization:

RNA was extracted from the transfected cells, using the RNeasy minikit (Quiagen®) according to the manufacturer's instructions The RNA was amplified using the Amino Allyl MessageAmp™ aRNA Amplification kit from Ambion [http://www.ambion.com/techlib/prot/fm_{—}1752.pdf]. Cy3 (green, absorption peak: 550 nm, emission peak: 570 nm) and Cy5 (red, 649/670 nm) fluorescent dyes were used for labeling. The RNA was fragmented to a length of ˜70-150 bp, by incubating the RNA samples with fragmentation buffer for 15 minutes at 70° C. The samples were pre-hybridized with pre-hybridization buffer (5×SSC, 0.1% SDS, 1% BSA), dried and hybridized (3×SSC, 0.1% SDS, 10 μg polyA, 20 μg tRNA) overnight at 65° C. The slides were then washed, dried, and analyzed.

Image processing was performed in a dedicated scanner (Affymetrix, 428 Array Scanner). Basic signal processing was determined using the ImaGene software. Data analysis was performed using the MatLab program, created by Dr. Yoram Ben-Shaul (Hebrew University of Jerusalem, Jerusalem, Israel).

TABLE 3
Sequences referred to in the present study:

| Sequence | Description |
|---|---|
| SEQ. ID. No. 1 | cDNA sequence corresponding to mE1a |
| SEQ. ID. No. 2 | cDNA sequence corresponding to mE1b |
| SEQ. ID. No. 3 | cDNA sequence corresponding to mE1c |
| SEQ. ID. No. 4 | cDNA sequence corresponding to mE1d |
| SEQ. ID. No. 5 | cDNA sequence corresponding to mE1d′ |
| SEQ. ID. No. 6 | cDNA sequence corresponding to mE1e |
| SEQ. ID. No. 7 | cDNA sequence corresponding to hE1a |
| SEQ. ID. No. 8 | cDNA sequence corresponding to hE1b |
| SEQ. ID. No. 9 | cDNA sequence corresponding to hE1c |
| SEQ. ID. No. 10 | cDNA sequence corresponding to hE1d |
| SEQ. ID. No. 11 | Protein sequence corresponding to the mE1e ORF |
| SEQ. ID. No. 12 | Protein sequence corresponding to the hE1d ORF |
| SEQ. ID. No. 13 | Peptide 437 |
| SEQ. ID. No. 14 | Peptide 438 |
| SEQ. ID. No. 15 | Protein sequence of hN-AChE-S (AChE-S sequence |
| | extended N-terminally by the stretch encoded by hE1d) |
| SEQ. ID. No. 16 | Protein sequence of hN-AChE-R (AChE-R sequence |
| | extended N-terminally by the stretch encoded by hE1d) |
| SEQ. ID. No. 17 | Protein sequence of hN-AChE-E (AChE-E sequence |
| | extended N-terminally by the stretch encoded by hE1d) |
| SEQ. ID. No. 18 | mRNA probe for exon mE1a |
| SEQ. ID. No. 19 | mRNA probe for exon mE1d |
| SEQ. ID. No. 20 | mRNA probe for exon hE1d |
| SEQ. ID. No. 21 | Forward PCR primer for exon mE1a |
| SEQ. ID. No. 22 | Forward PCR primer for exon mE1b |
| SEQ. ID. No. 23 | Forward PCR primer for exon mE1c |
| SEQ. ID. No. 24 | Forward PCR primer for exon mE1d |
| SEQ. ID. No. 25 | Forward PCR primer for exon hE1d |
| SEQ. ID. No. 26 | Forward PCR primer for mAChE-R |
| SEQ. ID. No. 27 | Forward PCR primer for mAChE-S |
| SEQ. ID. No. 28 | Forward PCR primer for mAChE-E |
| SEQ. ID. No. 29 | Reverse PCR primer for exons mE1a, mE1b, mE1c, |
| | and mE1d |
| SEQ. ID. No. 30 | Reverse PCR primer for exon hE1d |
| SEQ. ID. No. 31 | Forward PCR primer for mAChE-R and mAChE-E |
| SEQ. ID. No. 32 | Forward PCR primer for mAChE-S |
| SEQ. ID. No. 33 | Mouse AChE signal peptide |
| SEQ. ID. No. 34 | Human AChE signal peptide |
| SEQ. ID. No. 35 | mRNA probe for exon mE1b |
| SEQ. ID. No. 36 | cDNA sequence encoding hN-AChE-S |
| SEQ. ID. No. 37 | cDNA sequence encoding hN-AChE-R |
| SEQ. ID. No. 38 | cDNA sequence encoding hN-AChE-E |
| SEQ. ID. No. 39 | Signal peptide |

ORF = open reading frame.

### Example 1

### 6′ Diversity of Murine AChE mRNAs

EST database searches using the 5′ region of the mouse (m) ACHE gene revealed the existence of five putative alternative first exons (Table 4A, FIG. 1A). The most proximal exon was termed mE1a. The EST clone containing this sequence (GenBank Accession No. BB606349, mouse eyeball) extends from position −787 to −680 (relative to the translational ATG start present in the mouse exon 2) and continues with exon 2 (FIGS. 1A, 1B), skipping over a 657-nucleotide long intron (termed mouse mI1a) that possesses consensus GT-AG splice sites. RT-PCR and sequencing confirmed the existence of this transcript (GenBank Accession No. AY389982).

A second first exon, named mE1b, was found by RT-PCR using a forward primer located in the −945 to −923 region with a reverse primer on exon 2 (Table 2). The resulting product extends from this primer to position −733 and skips over a 710-nucleotide long intron (mI1b), which includes consensus GT-AG splice sites (FIGS. 1A, 1B). This exon, as well, was confirmed by sequencing (GenBank Accession No. AY389981).

Upstream to mE1b, at −1762 to −1671, the inventors found the ‘classical’ exon 1 [Li, Y. et al. (1991) J Biol Chem. 266, 23083-23090], renamed here mE1c, in 18 different reported homologous EST clones (GenBank Accession No. BB639234, Table 4A). When this first exon is fused to exon 2, a 1648-nucleotide intron (mI1c) that contains consensus GT-AG splice sites is spliced away. Sequencing of an RT-PCR amplified DNA fragment, confirmed the existence of mE1c.

An additional mRNA transcript that contains mE1c but proceeds through genomic sequence was mE1c-long. Two longer ESTs indeed initiated at mE1c (GenBank Accession Nos. BB629342 and CA327701, adult bone and whole brain embryo, respectively), and extend through the entire genomic sequence to exon 2 (GenBank Accession No. AK036443, adult male bone). In these ESTs, exon 2 is fused to exon 3. Splicing of intron 2 rules out the possibility of genomic DNA contamination as the source of this mE1c-long variant.

Further upstream, an alternative first exon [Atanasova, E. et al. (1999) J Biol Chem 274, 21078-21084] was previously found at position −2271 to −1980, followed by a 1957-nucleotide long intron (mI1d). This first exon was found to be fused with exon 2. The inventors confirmed the expression of the corresponding transcript in the prefrontal cortex (PFC) by RT-PCR and sequencing. This exon was named mE1d. Two alternative splice donors that differ by 29 nucleotides were observed. The shorter form was named mE1d' (GenBank Accession No. AY389980).

Upstream from mE1d, three putative different ORFs (positions −2518 to −2402, −2925 to −2522 and −3129 to −2933) were found in a continuous reading frame with that of the classical protein. These could potentially add 46, 142 or 73 amino acids (respectively) to the common ORF beginning at exon 2. Of these, the mE1e ORF shares 79% sequence similarity with the corresponding region in the human ACHE gene and its translated sequence (see below) and was thus regarded as a potential candidate. FIGS. 1A-1B depict the different mouse 5′ exons.

TABLE 4A
Alternative 5′ exons of mouse acetylcholinesterase

| | Number | Representative | Position* | Intron | Splice | | | |
|---|---|---|---|---|---|---|---|---|
| Exon | of ESTs | EST evidence | (from ATG) | size | Sites | ORF | Reference | Conf. |
| mE1a | 1 | BB606349 | 787 to 680 | 657 | GT-AG | No | — | Yes |
| | | Eye ball, PO | | | | | | |
| mE1b | 0 | — | 945 to 733 | 710 | GT-AG | No | — | Yes |
| mE1c- | 1 | AK036443 | 1762 to 22 | — | — | No | — | No |
| long | | Bone, adult | | | | | | |
| mE1c | 18 | BB639234 | 1762 to 1671 | 1648 | GT-AG | No | Li et al. | Yes |
| | | Thymus, P3 | | | | | (1993) | |
| mE1d | 0 | — | 2271 to 1980 | 1957 | GT-AG | No | Atasanova | Yes |
| | | | | | | | et al. (1999) | |
| mE1d′ | 0 | — | 2271 to 2008 | 1986 | GT-AG | No | — | Yes |
| mE1e | 0 | — | 2518 to 2403 | 2380 | GT-AG | Yes | — | No |

*For convenience, the (—) mark in front of all position numbers is not indicated.
Conf. = confirmation.

### Example 2

### 5′ Diversity of Human AChE mRNAs

EST database searches using the 5′ region of the human (h) ACHE gene revealed the existence of at least 4 alternative first exons (Table 4B). The previously identified mouse EST clone (mE1a GenBank Accession No. BB606349, see above) suggests the existence of the alternative first exon named hE1a.

The previously described first exon at −1681 to −1576 (relative to the translational start site ATG present in the human exon 2) [Ben Aziz-Aloya, R. et al. (1993) Prog Brain Res 98, 147-153] is named here hE1b (represented by EST clone BG707892, human brain hypothalamus). A 1543-nucleotide intron (hI1b) separates hE1b and exon 2. The inventors confirmed the existence of hElb by RT-PCR and sequencing.

An additional EST clone contained the genomic sequence located at position −1859 to −1824 (GenBank Accession No. BI667712, human brain hypothalamus). This putative first exon was named hE1c. Followed by an intron of 1803-nucleotide intron (hI1c), it is fused to exon 2 at position −20 (ACG). The corresponding intron includes donor and acceptor splice sites (GT-AG). In this case, exon 2 starts at a different position. This is explainable by the fact that exon 2 starts with 2 optional acceptor splice sites located 3 nucleotides apart (both AG dinucleotide, FIGS. 1C, D). Our attempts to confirm the existence of this transcript failed.

An additional EST clone (GenBank Accession No. BX420294, human fetal brain) contained a putative first exon located further upstream at position −2720 to −2318 (exon hE1d) fused with exon 2 at position −20. This implies the existence of a 2294-nucleotide intron (hI1d). Intriguingly, hE1d harbors a translation start codon (ATG, position −2495) creating a continuous reading frame with that of the ‘classical’ ATG in exon 2 [Soreq, H. et al. (1990) Proc Natl Acad Sci USA 87, 9688-9692], thus potentially adding 66 amino acids to the AChE protein. An additional ATG in the same ORF may yield a shorter 61 amino acids domain. Sequence homology with mE1e, which lacks the first ATG, suggests the second ATG that is more likely to serve as the translational start site. The inventors confirmed the existence of this mRNA by RT-PCR and sequencing (GenBank Accession No. AY389977, FIGS. 1C, D).

TABLE 4B
Alternative 5′ exons of human acetylcholinesterase

| | No. of | Representative | Position* | Intron | Splice | | Exon 2 | | |
|---|---|---|---|---|---|---|---|---|---|
| Exon | ESTs | EST evidence | (from ATG) | size | Sites | ORF | start | Ref. | Conf. |
| hE1a | 1 | BB606349 | 768 to 732 | — | — | No | — | — | No |
| | | Eye ball, PO | | | | | | | |
| hE1b | 23 | BG707892 | 1681 to 1576 | 1543 | GT-AG | No | CAG | Ben- | Yes |
| | | Hypothalamus | | | | | | Aziz | |
| | | | | | | | | Aloya et | |
| | | | | | | | | al. | |
| | | | | | | | | (1993) | |
| hE1c | 1 | BI667712 | 1859 to 1824 | 1803 | GT-AG | No | ACG | — | No |
| | | Hypothalamus | | | | | | | |
| hE1d | 3 | BX420294 | 2720 to 2318 | 2294 | GT-AG | Yes | ACG | — | Yes |
| | | Fetal brain | | | | | | | |

*For convenience, the (—) mark in front of all position numbers is not indicated.
Conf. = confirmation.
Ref. = reference

### Example 3

### Putative Promoters for the Novel Exons

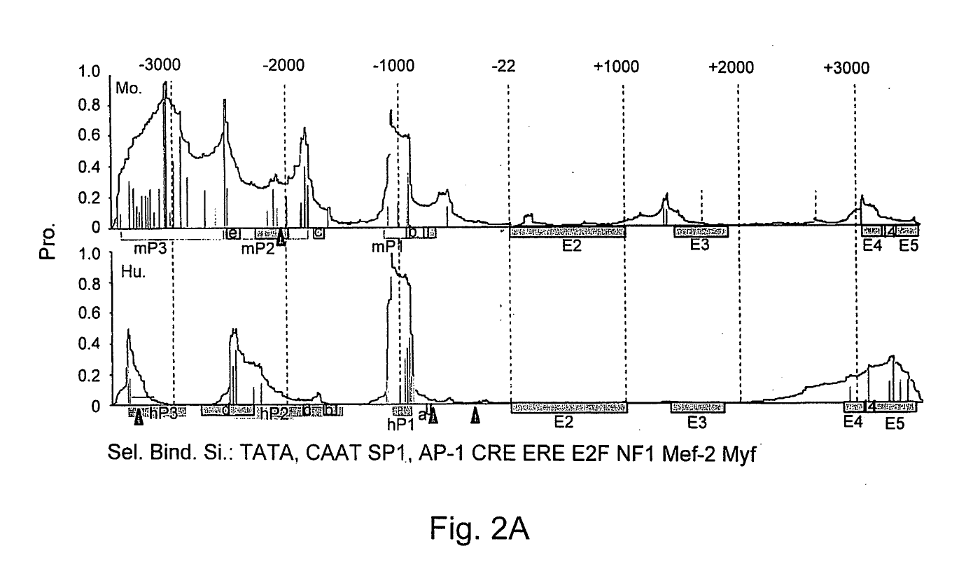

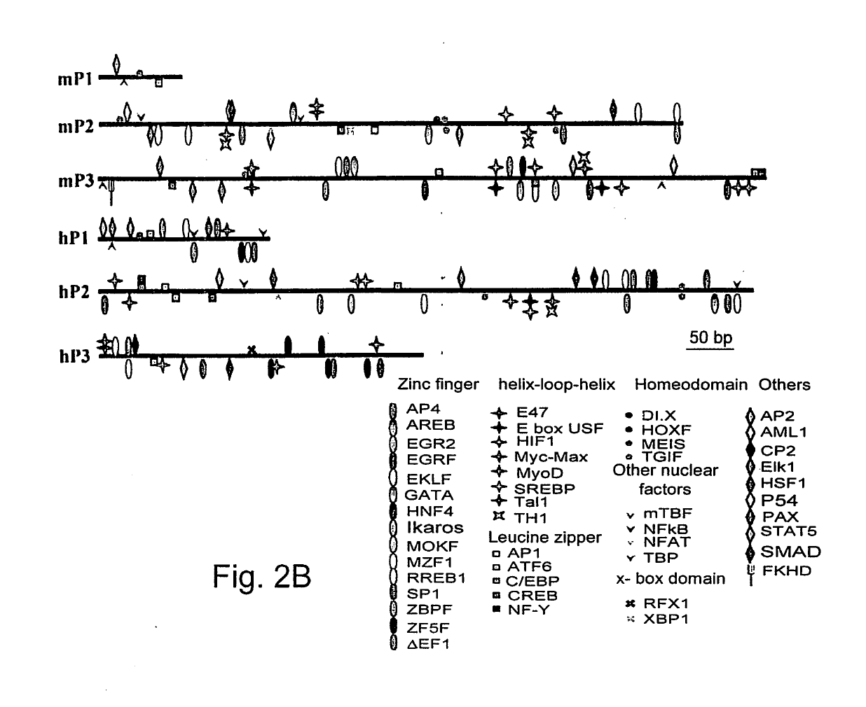

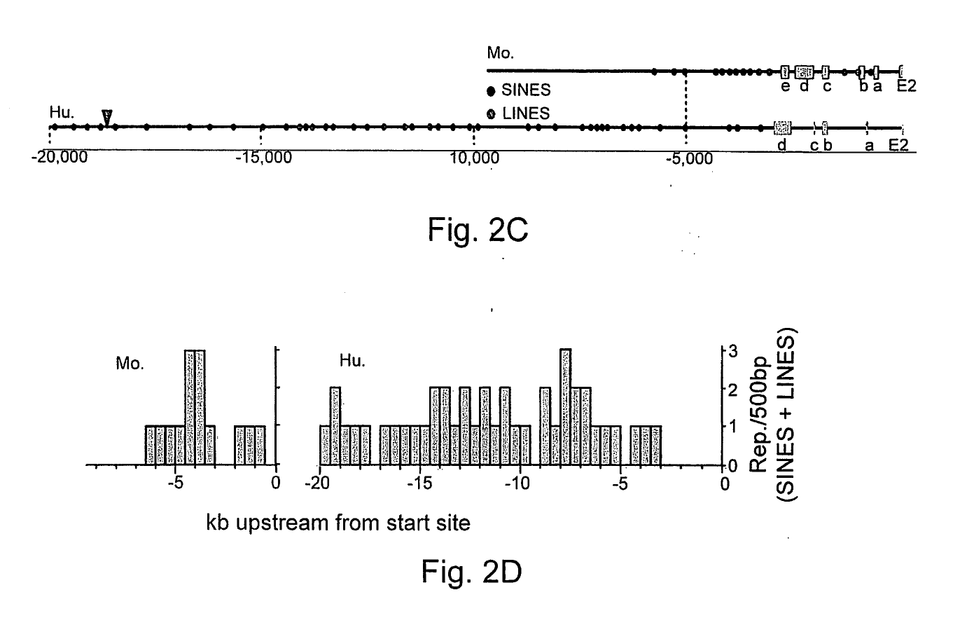

Using luciferase assays, Atanasova [Atanasova (1999) id ibid.] demonstrated the functionality of the promoter located upstream to mE1d (referred to in their work as exon E1a). In our study, the Cister (zlab.bu.edu/˜mfrith/cister.shtml) and Chip2Promoter (genomatix.de) programs enabled promoter predictions. These programs search for regions with motifs conservation predicting higher probability to be transcriptionally active promoters, shown in FIG. 2A for the murine and human ACHE genes. Based on the density of putative transcription factor binding sites, several regions with a higher probability to be a promoter were revealed by this search. These were located in the genomic regions upstream from the second exon of both the mouse and human genes (FIG. 2A). Promoter prediction analyses of the region containing the novel alternative first exons revealed a plausible promoter for each of the newly identified exons (FIGS. 2A, 2B). It is worth noticing that the probability of the alternative promoters is similar to that of the previously described promoter (upstream to mE1b in mouse and hE1b in human), supporting the notion that they might be functionally active. A particularly high probability to function as a promoter was observed for the mouse region upstream to exon mE1a. In the human gene, the inventors identified hE1a based on homology to the mouse mE1a. Exon hE1a is a weak candidate for being a true exon since it lacks consensus splice sites and since no ESTs were found in the entire region between exon 2 and exon hE1b in the human sequence. However, the region located upstream to hE1a displays the highest probability to function as a promoter (FIG. 2A), perhaps suggesting functionality that was lost during primate evolution. The Cister and Chip2Promoter programs, which do not apply for murine sequences, yielded similar predictions for human promoters.

A closer look at the distribution of the transcription factor binding sites revealed only a few which are unique to one out of the putative alternative promoters, and evolutionarily conserved in both human and mouse. Several putative DNA targets for transcription factors that respond to different signaling pathways were found: a conserved binding site for the transcription factor Dlx, highly expressed during organ development [Panganiban, G. and Rubenstein, J. L. R. (2002) Development 129, 4371-4386], was found in mP1 and hP1, and a putative binding site for TGIF in mP2 and hP2. Of interest, three putative glucocorticoid response elements (GREs) were identified on the upstream region of the human ACHE gene (one in hP3 and one adjacent to hE1a, FIG. 2A), and one such site was identified on the mouse gene (mP2, FIG. 2A). It was therefore tempting to further check whether some of the newly identified transcripts may be indeed glucocorticoid and/or stress responsive.

### Example 4

### Human and Mouse Syntheny

The upstream human and mouse sequences were scanned for homologous regions using the blast-2-sequences program (www.ncbi.nlm.nih.gov/blast). Seven homologous regions of different lengths were found (FIG. 2C). These include a short region adjacent to exon 2, mouse and human hE1a, a 270-bp region (corresponding to the strong promoter region upstream to exon 1) harboring part of mE1b (no corresponding exons were identified in the human sequence in this region), a 125-bp region which includes neither exons nor predicted promoters, the two ‘classical’ exons (hE1b and mE1c), a short sequence adjacent to hE1c and mE1d and a relatively long sequence showing homology between human hE1d and mouse mE1e. This pronounced homology, and the ORFs with similar features in hE1d and mE1e strengthen the plausibility of a common evolutionarily conserved ancestor sequence and the yet un-validated mE1e.

### Example 5

### SINEs and LINEs Separate 5′ Alternative Exons from the Distal Human ACHE Promoter

Alu repeats are the most abundant short interspersed elements (SINEs) within the primate genome [Batzer, M. A. and Deininger, P. L. (2002) Nat Rev Genet 3, 370-379]. In humans, 1.5 million SINEs account for some 13%, and the 850,000 long interspersed elements (LINEs) for another 21%, comprising together a grand total of 34% of the genome [Weiner, A. M. (2002) Curr Opin Cell Biol 14, 343-350]. LINEs are usually found in gene-poor, AT-rich areas; SINEs are preferentially located within gene-rich regions, reflecting preferred availability for insertion events, but usually not inside exons, where such insertions may interfere with expression [Batzer and Deininger (2002) id ibid.]. On average, one might expect one SINE and one LINE for approximately every 2-3.5 kb, except within the transcription unit itself. A totally different outcome emerged for the currently available GenBank sequences (20 of the human, GenBank Accession No. AF002993, and 9.5 kb of mouse, GenBank Accession No. AF312033) upstream to the translation start site of exon 2. The SINEs and LINEs distribution in the analyzed sequences was analyzed using the Eldorado software (genomatix.de) and the RepeatMasker algorithm (searchlauncher.bcm.tmc.edu). The density is 6-fold higher than average for SINEs and almost 2-fold higher than average for LINEs. This leaves little room for any functional DNA in this area. In contrast, exceptionally few repeats were found within the human and mouse 3.5 kb regions where the alternative first exons were identified (1 and 3 repeats, respectively), supporting a functional role for these DNA fragments in human and mouse. The closest gene upstream to ACHE is located approximately 180 kb away [Wilson, M. D. et al. (2001) Nucleic Acids Res 29, 1352-1365].

### Example 6

### Tissue Distribution of the Novel Exons in Mouse

Tissue distribution in mouse of the mRNAs containing the different alternative first exons was studied by RT-PCR (FIG. 3A). Exon mE1a was found to be expressed in every examined brain region, including hippocampus, cortex, PFC, brainstem and basal nuclei. Exon mE1a was also expressed in the thymus, heart, liver, intestine, and spleen, but not in kidney, testis, muscle, or spinal cord. Exon mE1b was detected in most of the tissues examined, with the exception of liver, intestine and muscle. Exon mE1c was the most widely expressed. It was, however, absent from intestine. Exon mE1d was detected in the brain (hippocampus, PFC, brainstem and basal nuclei) and heart, but not spleen, thymus, intestine or liver. For comparison, the inventors investigated in the same tissues the expression profiles of the different AChE 3′ variants. ‘Synaptic’ AChE-S was strongly expressed in all tissues examined, except for thymus, liver and the small intestine, where only weak expression was observed. It could be predicted, therefore, that the most common 5′ transcript, the ‘classic’ mE1c would be the primary partner of AChE-S in the mature AChE-S mRNA variant. Nevertheless, an alternative 5′ transcript should form the mature AChE-S mRNA variant in the intestine, where mE1c is not expressed. ‘Read-through’ AChE-R was strongly expressed in all of the brain regions tested and in the spleen. It was moderately expressed in heart, muscle, kidney, spinal cord and liver, and very poorly expressed in the testis, thymus and intestine. ‘Erythrocytic’ AChE-E was expressed in all of the examined brain regions as well as in heart, kidney, spinal cord, liver, spleen, and muscle. It was absent from testis, thymus and the small intestine. Thus, none of the 5′ variants shared the same expression pattern with a single 3′ variant, suggesting that 5′ splicing patterns do not always dictate 3′ splicing in the mature mRNA. The four different 5′ and three different 3′ splice options may thus yield 12 distinct transcripts.

### Example 7

### Distinct Neuronal Distributions of the 5′ Murine Exons

To achieve cellular resolution levels for the expression patterns of the novel exons, the inventors designed 40 to 50-mer 5′-biotinylated fully 2′-β-methylated riboprobes for fluorescent in situ hybridization (FISH, see Experimental Procedures for details). FIG. 3B presents representative FISH profiles for mE1a, mE1b and mE1d.

These three exons all appeared to be expressed in neurons. They displayed, however, distinct cell type specificities and subcellular distributions. For example, principally all of the deep layer PFC neurons displayed pronounced mE1a levels and considerably lower mE1b labeling. Exon mE1d mRNA was particularly concentrated in the uppermost layer of PFC neurons (FIG. 3B1), suggesting distinct levels for this variant in specific subsets of PFC neurons. Whereas these differences could potentially reflect probe efficiencies, they indicate that the various alternative mRNAs have distinct expression patterns. Hippocampal CA2 neurons within the same or adjacent sections displayed consistently low levels of all three exons (FIG. 3BII), supporting the notion of these cell type differences. Differential expression of the various 5′ exons was also conspicuous in cerebellar neurons (FIG. 3BIII). mE1a accumulated in the cytoplasm of Purkinje cell perikarya but was only faintly detected in other cerebellar neurons. mE1b was poorly expressed in the cerebellum, and mE1d was strongly expressed in Purkinje cells, in which it was labeled in both cell bodies and axonal processes (FIG. 3BIV, V). In addition, mE1d is transcribed in other neurons of the cerebellum, including the smaller cells interspersed in the molecular layer, where it displays an asymmetric labeling pattern. In these neurons, neurites were also labeled. Granular neurons were only poorly labeled with the probe mE1d.

### Example 8

### Human hE1d mRNA Expression—Embryonic Expression

The tissue distribution of hE1d mRNA in later developmental stages was explored in paraffin sections from human embryos aged 16, 25 and 34 weeks. At week 16, hE1d mRNA was only weakly detected in the nervous system and was absent in the thymus. As development proceeded, hE1d expression became more pronounced, with increased density of positive cells and increased labeling intensity in both the nervous system and the thymus. At week 34, up to 50±10% of the neurons were positive (FIG. 4A). In contrast, as low as 2±1.5% of the thymus cells were hE1d mRNA positive at week 25, but by week 34, over 8±1.5% of the cells were positive.

Human hE1d mRNA Expression—Adult Expression

FISH analysis of paraffin-embedded human PFC sections revealed prominent neuronal hE1d mRNA labeling, with 57±34% of the cells in the PFC being hE1d mRNA-positive. Up to 25% of the labeled cells displayed hE1d mRNA labeling in neuritic processes, reaching 14.5±7.5 μm in length (FIG. 6D).

### Example 9

### Stress and Glucocorticoid-associated Expression of the Novel Exons

Stress induces rapid [Kaufer, D. et al. (1998) Nature 393, 373-377] yet long-lasting [Meshorer (2002) id ibid.] expression of AChE-R mRNA encoding an AChE variant with a cysteine-free C-terminus, which leads to the accumulation of stress-associated AChE monomers. The ACHE gene possesses a GRE in a distal enhancer [Shapira (2000) id ibid.], and ACHE gene expression increases following corticosterone administration [Meshorer (2002) id ibid.]. The inventors therefore investigated whether any of the novel 5′ exons are selectively over-produced following stress in control mice as compared with mutant mice that selectively lack the GR gene in their central nervous system (GR^{NesCre} mice), [Tronche (1999) id ibid.].

In the mouse PFC, mE1b mRNA levels were unaltered in the GR^{NesCre} animals as compared with controls. However, when the mutant animals were stressed by immobilization, mE1b mRNA decreased significantly within 2 hr in GR^{NesCre} mice as compared with either unstressed GR^{NesCre} mice or with stressed control mice (FIGS. 5A-5B), implying a role for the GR in maintaining normal levels of mE1b following stress. In contrast, mE1c mRNA levels increased similarly in stressed control and GRNescre animals. This suggests that the expression of the mE1c exon is up-regulated in response to immobilization stress in a manner which does not involve the GR transcription factor. Mouse mE1d, however, was markedly up-regulated 2 hr after immobilization stress in control mice, but only very slightly in GR^{NesCre} animals. This suggests massive stress-induced and glucocorticoid-dependent regulation of mE1d. AChE-S mRNA remained generally unchanged in stressed wild type mice, compatible with our previous findings [Kaufer (1998) id ibid.; Meshorer (2002) id ibid.]. In contrast, AChE-S mRNA levels decreased substantially in stressed mutant mice, suggesting that the 3′ alternative splicing pattern of AChE pre-mRNA is glucocorticoid dependent. Thus, while actin mRNA levels remained unchanged, each of the analyzed variant exons displayed a unique combination of stress and glucocorticoid responses.

### Example 10

### N-AChE Protein Products and their Expression

Novel N-terminal putative ORFs, in frame with the AChE coding sequences, were identified in orthologous regions of the mouse mE1e and the human hE1d exons. The putative ORF of mE1e encodes 46 additional amino acids, a domain with no homology with any known protein in the database (FIG. 6A). These include 8 positively charged residues (4 arginines, one lysine and 3 histidines), but only 2 negatively charged ones (2 glutamates), yielding an extremely high pI value of 11.54. Secondary structure analysis of mEle (GOR4 software http://npsa-pbil.ibcp.fr/cgi-bin/secpred_gor4.p1) revealed a potential alpha helical folding (FIG. 6B, top). The mE1c-encoded peptide was analyzed by the Motif Scan software (http://hits.isb-sib.ch/cgi-bin/PFSCAN, available us.ExPASy.org) revealing a putative protein kinase phosphorylation site (position 4-6, TsR), and an N-myristoylation site (position 13-18, GGhrSG, FIG. 6B). An addition of this peptide chain to the N-terminus will most likely prevent cleavage of the mouse AChE signal peptide (MRPPWYPLHTPSLAFPLLFLLLSLLGGGARA, positions 1-31, SEQ. ID. No.33). This will yield a 77 (46+31) amino acids extension of the mN-AChE protein (13.4% increase over the 574 residues of mAChE-S, [Rachinsky, T. L. et al. (1990) Neuron 5, 317-327], with the signal peptide predicted to become transmembranal (e.g. the asialoglycoprotein receptor variant, [Spiess, M., and Lodish, H. F. (1986) Cell 44, 177-185].

The corresponding human exon hE1d encodes for an N-terminal extension of 66 amino acids, in frame with the hAChE protein (FIG. 6B). This peptide as well precedes the human AChE signal peptide (MRPPQCLLHTPSLASPLLLLLLWLLGGGVGA, position 1-31, SEQ. ID. No.34) that is normally cleaved off during maturation. The inventors predicted its presence to prevent AChE cleavage, resulting in a larger protein of 92 (61+31) or 97 (66+31) amino acids, 16-17% increase over the 574 residues of AChE-S [Soreq (1990) id ibid.].

No significant homology was found for the hN-AChE peptide sequence in the SwissProt database. Similar to mN-AChE, the peptide includes a putative phosphorylation site (for casein kinase II, position 7-10, ScpD), as well as an N-myristoylation site (position 31-36, GGsrSF, FIG. 6A). In addition, similar to mN-AChE, hN-AChE displays an extremely high predicted pI (11.76), similar to that of histones and other nucleic acid binding proteins (http://www.expasy.org/tools/tagident.html).

Anti-hN-AChE antibodies recognized, in immunoblots of glioblastoma protein extracts, a 66 Kd double band, comparable to the labeling pattern observed using the N19 anti-AChE antibody (FIG. 6C, inset, top left). Protein extracts from different regions of the human brain (shown schematically in FIG. 6E) demonstrated a similar size for the hN-AChE protein in vivo (FIG. 6C, bottom). Expression spanned various cortical domains, including PFC and the occipital cortex, where it was most prominent. The hippocampus, striatum and amygdala were also positive, but cerebellar expression was very low. These results, together with the mRNA expression analysis described in Example 8, show that a significant fraction of the stress-responding PFC neurons thus express hN-AChE both in their cell body and in neurites

Rabbit polyclonal antibodies were generated against two short internal peptides from the hN-AChE ORF (FIG. 4B), and used in flow cytometry analysis to identify hematopoietic cells expressing hN-AChE. Although unsatisfactory for immunohistochemistry on paraffin-embedded sections, the anti-hN-AChE antibodies successfully labeled cells of human cord blood. Cell lineages were classified according to their relative side scatter and their expression levels of the blood cell marker CD45. Five different clearly distinguishable populations were detected: lymphocytes (L), monocytes (M), granulocytes (G), blood cells progenitors (P), and nucleated erythrocytes (NE, FIG. 4C1). Monocytes and granulocytes displayed the most prominent labeling, with 67±19 and 57±21% of the cells expressing hN-AChE, as compared to an isotype control. In addition, 17±7% of the lymphocytes and 7.5±4% of CD34+ progenitors were hN-AChE-positive, while nucleated erythrocytes were completely negative (FIG. 4CII). To further subclassify the lymphocytes expressing hN-AChE, specific markers for stem cells (CD34), early lymphocytes (IL7), mature T-cells (CD3) and mature B-cells (CD19) were used. While part of these markers may appear in more than one cell lineage, T-cells were the most prominent, with 9±3% CD34+ lymphocytes, rising to 10±3% positive early T-cells and increasing to 14±9% in mature T-cells. B-cells, as well, were 7.5±6.5% hN-AChE positive.

To test whether hN-AChE is expressed in the membrane, as predicted from its primary structure, the flow cytometry tests were repeated following permeabilization of the cells. No increase was observed following permeabilization; rather, monocyte and granulocytes labeling decreased to 7±1% and 20±7.5, respectively, implying that hN-AChE is expressed in the membrane.

### Example 11

### N-AChE is Overexpressed in Alzheimer's Disease

AChE activity is known to decrease late in the course of Alzheimer's disease (AD), which likely contributes to the pathogenesis of this disease. However, the composition in AD of specific AChE variants remained unknown. To address this question, the inventors performed fluorescent in-situ hybridization (FISH) with cRNA probes complementary to exon 6, pseudo intron 4 and the novel 5′ exon E1d to detect AChE-S, AChE-R and N-AChE transcripts.

An antibody against the core domain of AChE, common to all known variants, reveals massive down-regulation of total AChE levels in dentate gyrus neurons (p<0.05) (FIG. 9A-9C), suggesting a massive decrease in the normally prevalent AChE-S protein.

FISH mRNA labeling in dentate gyrus neurons showed a clear decrease in the levels of the ‘synaptic’ (AChE-S) variant (FIGS. 10A and 10C) and a modest but significant increase in the levels of the ‘readthough’ (AChE-R) variant (* p<0.01, **p<0.05 Student's t-test) (FIGS. 10B and 10C), changing the ratio between these two variants and increasing the production of the normally rare AChE-R form. Parallel increase in the levels of AChE-R mRNA has been observed in double transgenic mice expressing both mutated APP and human AChE-S in excess [Rees, T. M. et al. (2005) Current Alzheimer Research In press].

Using a probe specific to E1d, a significant increase in the corresponding mRNA transcript was observed in the dentate gyrus of an Alzheimer's Disease specimen (FIGS. 11A and 11C) as compared to CA3 neurons of the aged human hippocampus, either control or Alzheimer's disease (FIGS. 11B and 11C).

FIGS. 12A and 12B show immunolabeling of the hippocampus using antibodies specific to the N′ terminus (which detects the N-AChE variant) or to the C′ terminus (which detects the AChE-S variant). The labeled region revealed upregulation of the N-AChE-S variant in the mossy fiber system, which connects the dentate gyrus to the CA3 neurons region, in Alzheimer's disease.

The expression of all AChE isoforms (AChE-S, AChE-R and N-AChE) in the hippocampus was confirmed through RT-PCR (FIG. 13).

Thus, major changes in the composition of AChE variants were observed in the human Alzheimer's disease hippocampus. These changes were detected both at the mRNA and at the protein levels, suggesting that altered regulation of the ACHE gene expression is a key feature of Alzheimer's disease. Changes involve altered promoter usage, modified alternative splicing and changed location of AChE in the AD brain. These changes probably have considerable effects on synaptic transmission or even on neuronal cell death, as AChE has been reported to induce apoptosis [Zhang (2004) id ibid.], or beta-amyloid aggregation, as AChE, is one of the amyloid plaque components, and was shown to facilitate beta-amyloid fibrillation [Inestrosa (1996) id ibid.].

### Example 12

### Overexpression of AChE-R or AChE-S Results in Altered Gene Expression Profile

The inventors set on to identify transcriptional and post-transcriptional changes involved in alternative splicing and/or apoptosis occurring in transfected cells overexpressing specific AChE variants. Using an in-house microarray enabled the identification of candidate genes that are affected by overexpression of AChE-R or AChE-S in the p19 embryocarcinoma cell line.

P19 cells were treated for 3 days with 0.5 μM of retinoic acid [Jones-Villeneuve, E. M. et al. (1982) J Biol Chem 94(2): 253-62], which is known to induce the differentiation of these cells into the neuronal lineage. On day 4 cells were transfected with 1 μg of one of the following vectors: a vector overexpressing AChE-S [Ben-Aziz Aloya, R. et al. (1993) Proc. Natl. Acad. Sci. USA 90: 2471-2475]; a vector overexpressing AChE-R [Seidman, S. et al. (1995) Mol Cell Biol. 15: 2993-3002]; or an empty vector for control. The cells were partially differentiated, and showed elevated levels of choline acetyl transferase (ChAT), while showing relatively high levels of transfected DNA. On day 5 RNA was extracted from the transfected cells, using the RNeasy minikit (Quiagen®) according to the manufacturer's instructions. RNA from cells over-expressing each vector was compared to RNA from cells transfected with the empty vector. In addition, dye-swapping tests were performed, aimed at excluding those labeling differences that are due to the different dyes employed. Such comparisons were comprised, for each experimental sample, of 4 different slides, according to the following:

### Slide Sample

1 Experimental labeled with Cy3/Control labeled with Cy5
2 Experimental labeled with Cy3/Control labeled with Cy5
3 Experimental labeled with Cy5/Control labeled with Cy3
4 Experimental labeled with Cy5/Control labeled with Cy3

Slides 1 and 2, and 3 and 4 are identical replicates. In addition, this design employed “dye-swapping”, i.e. each sample was labeled twice with one fluorescent dye and twice with the other, in order to compensate for dye-specific effects, which are known to occur in microarray staining assays.

The RNA was amplified using the Amino Allyl MessageAmp™ RNA kit from Ambion [http://www.ambion.com/techlib/prot/fm_{—}1752.pdf] Cy3 (green, absorption peak: 550 nm, emission peak: 570 nm) and Cy5 (red, 649/670 nm) fluorescent dyes were used for labeling. RNA fragmentation, pre-hybridization and hybridization were performed as described in the Experimental Procedures.

FIGS. 16A-16C and 17A-17I show the results of the microarray analysis of P19 cells overexpressing AChE-R or AChE-S. The results may be summarized essentially as follows. AChE-R or AChE-S had three main effects on gene expression:

1) One group of genes was regulated similarly by the two AChE isoforms (either induced or inhibited, but the same result for both treatments), suggesting that the changes in expression pattern are related to the common protein domain or to the catalytic activity of AChE.
2) One group of genes (or gene families) that up- or down-regulated by one of the isoforms and has the opposite effect by the other.
3) Individual genes whose expression is changed by one of the isoforms and is unchanged by the other.
4) Other genes showed no effect by any of the transfected DNAs, demonstrating selectivity of their effects.

Generally, three main groups of genes were affected by the overexpression of AChE-R/S: apoptosis-related, helicases, and SR and SR-related genes. Interestingly, SR and SR-related genes are mostly dwnregulated by both isoforms, whereas apoptosis-related genes were upregulated by AChE-R and downregulated by AChE-S (although the analysis did not differentiate between pro-apoptotic and anti-apoptotic genes). Expression of the helicase genes changed only in AChE-S expressing cells. This result may be correlated with the inventors' preVious results showing nuclear localization of AChE-S in the nucleus [Perry et al. (2002) Oncogene. 21(55):8428-41].

1. A cDNA sequence derived from the ACHE gene, comprising a variant 5′ region.

2. The cDNA sequence of claim 1, wherein said ACHE gene may be from mouse or human origin.

3. A cDNA sequence comprising an AChE variant sequence at its 5′ end, wherein said variant sequence is substantially as denoted by any one of SEQ. ID. No.1, 2, 3, 4, 5, 6, 7, 8, 9 and 10, as well as functional analogues and derivatives thereof.

4. A peptide encoded by a nucleic acid sequence derived from the ACHE gene, wherein said peptide comprises AChE transmembrane and intracellular domains.

5. The peptide of claim 4, wherein said ACHE gene may be from mouse or human origin.

6. The peptide of claim 4, denoted by any one of SEQ. ID Nos. 11 and 12, as well as functional analogues and derivatives thereof.

7. A peptide derived from the human ACHE gene, wherein said peptide comprises the sequence substantially as denoted by any one of SEQ. ID. Nos.12, 13 and 14, as well as functional analogues and derivatives thereof.

8. A peptide derived from the mouse ACHE gene, wherein said peptide comprises the sequence denoted by SEQ. ID. No.11, as well as functional analogues and derivatives thereof.

9. A peptide derived from the human AChE transmembrane domain, wherein said peptide is substantially as denoted by any one of SEQ. ID. Nos.13 and 14, as well as functional analogues and derivatives thereof.

10. An AChE protein comprising a transmembrane domain.

11. The AChE protein of claim 10, wherein said AChE is one of the -S, -R and -E forms, denoted by sequences SEQ. ID. Nos. 15, 16 and 17, respectively, as well as functional analogues or derivatives thereof.

12. A nucleic acid construct comprising any one of the sequences denoted by SEQ. ID. Nos. 1-10 and 36-38, operably linked to at least one control element.

13. A transfected cell containing an exogenous sequence, wherein said cell is transfected with one of the construct of claim 12, a cDNA sequence derived from the mouse or human ACHE gene, comprising a variant 5′ region, or a cDNA sequence comprising an AChE variant sequence at its 5′ end, wherein said variant sequence is substantially as denoted by any one of SEQ. ID. No.1, 2, 3, 4, 5, 6, 7, 8, 9 and 10, as well as functional analogues and derivatives thereof.

14. A sensor for a cholinergic signal, wherein said sensor comprises the AChE extracellular, transmembrane and intracellular domains.

15. The sensor of claim 14, wherein said AChE transmembrane and intracellular domains comprise the sequence as denoted by one of SEQ. ID. Nos. 11 and 12.

16. A cell expressing a AChE transmembrane domain for use as a sensor for one of stress and cholinergic imbalance.

17. An AChE protein, wherein said protein is denoted by one of sequences SEQ. ID. Nos. 15, 16 and 17, as well as derivatives thereof, and wherein said protein is secreted.

18. An AChE protein, wherein said protein comprises at its N-terminus the sequence denoted by SEQ. ID. No.39 and it is secreted.

19. An AChE protein comprising a transmembrane domain and/or intracellular domain, wherein said protein is a derivative of AChE in which one or both of said domains has at least one deleted, inserted or substituted residue.

20. The AChE protein of claim 19, wherein said protein is secreted.

21. A method of recombinantly producing an AChE protein, said method comprising preparing a culture of recombinant host cells transformed or transfected with a recombinant nucleic acid molecule encoding an AChE protein or with an expression vector comprising said recombinant nucleic acid molecule; culturing said host cell culture under conditions permitting the expression of said protein; and recovering said protein from the cells.

22. The method of claim 21, wherein said nucleic acid molecule is denoted by one of SEQ. ID. No. 36, SEQ. ID. No. 37 and SEQ. ID. No.38.


SEQUENCE LISTING (39 sequences)

SEQ ID NO: 1 - DNA, 110 bp, Mus musculus
cacaccaaggctcgggcggggagctctggcctcttcttggtctctactgctcccggttggcagcggagggcattgcaatatggggatgcaataggggcttgagttctggt

SEQ ID NO: 2 - DNA, 214 bp, Mus musculus
ttttgatctcttggctggagacgccggaactacagcagctgttgcccccaaaatagcgcccctgcctttgctacggggatctccggagctcccggaacacagacgtcctggctcgcccttcaaccccctctgcgatgctcacgattctccagatacccccacaccaaggctcgggcggggagctctggcctcttcttggtctctactgctcccg

SEQ ID NO: 3 - DNA, 94 bp, Mus musculus
cggctgtcactgtcggctcagcctgcgccggggaacattggccgcctccagctcccggcgcggcccgacccggcccggcttggccgcctcaggt

SEQ ID NO: 4 - DNA, 276 bp, Mus musculus
caggtgttctttcgtctccacagagatgtcccacgtgtcagctgaagggggtcctcagtcaggctttctcgtgtctcttctcttattaccctgccccaagctttgtcctggttacagatgccaaatattaggcctctgatctttctggattagagctgtcagtgtgtccttccgtccgtgaaaggcgaccggtctgtctgtgacttgtcaccgcaggagactgtcgcctgcgtgaacggtgtctggtgtctccgcaggctgcggtccgtctgtcac

SEQ ID NO: 5 - DNA, 263 bp, Mus musculus
caggtgttctttcgtctccacagagatgtcccacgtcatcttttctaccagtgtcagctgaagggggtcctcagtcaggctttctcgtgtctcttctcttattaccctgccccaagctttgtcctggttacagatgccaaatattaggcctctgatctttctggattagagctgtcagtgtgtccttccgtccgtgaaaggcgaccggtctgtctgtgacttgtcaccgcaggagactgtcgcctgcgtgaacggtgtctggt

SEQ ID NO: 6 - DNA, 190 bp, Mus musculus
caggctgggggctccaagtgtaggccgctcccacctgctagccacaacaaccaatgaaagcttggcccggtgatgtcatggaccagccggacccttgtgatgacagccggaggtcaccggtcaggggaattagctcaagcccaccctcgggggttccggaagtttccagagctgcagcagagtgcattgt

SEQ ID NO: 7 - DNA, 38 bp, Homo sapiens
ggggcggggagcgccgaccttttctcggtctctactgc

SEQ ID NO: 8 - DNA, 113 bp, Homo sapiens
tgtgcggggggccggaggcggcggctgtcagagtcggctcagcctgcgccggggaacatcggccgcctccagctcccggcgcggcccggcccggcccggctcggccgcctcag

SEQ ID NO: 9 - DNA, 42 bp, Homo sapiens
actgcgctctgtcgtcggtcggcgtctggcactggagtgcgt

SEQ ID NO: 10 - DNA, 369 bp, Homo sapiens
cagggagcaggcgccgcgcggtattgccgcatgcacctcggtcgtggggaccccgctgcagcagccctgtccacacgggtgaactcctggagggcagggcggggggcaatctgcgcgggtcaggcccctcggagcaggctgggggcgccgagccaggccgctcccacctgccagccgcgtggccaatgaatgctaggcctggtgatgtcatgccccgaccggaccctggtgacgaaagtccgaagtcacccgtcagggaaccagcacagacccacccgcgggggttccagaagtttccactgccgccgcggagtgcggcctcgcccagcagccttgcgcgtgctaccacgctgtcctgccttctcaggt

SEQ ID NO: 11 - protein, 69 aa, Mus musculus
MLGLVMSWTSRTLVMTAGGHRSGELAQAHPTRGGSRSFHCRRGFRKFPELQQSALHQPVLSDAACPAVM

SEQ ID NO: 12 - protein, 69 aa, Homo sapiens
MLGLVMSCPDRTLVTKVRSHPSGNLAQHRPTRGGSRSFHCRRGVRPRPAALRVLPRCPAFSDAACPAAM

SEQ ID NO: 13 - protein, 16 aa, Homo sapiens
KVRSHPSGNQHRPTRG

SEQ ID NO: 14 - protein, 16 aa, Homo sapiens
GSRSFHCRRGVRPRPA

SEQ ID NO: 15 - protein, 681 aa, Homo sapiens
MLGLVMSCPDRTLVTKVRSHPSGNQHRPTRGGSRSFHCRRGVRPRPAALRVLPRCPAFSDAACPAAMMRPPQCLLHTPSLASPLLLLLLWLLGGGVGAEGREDAELLVTVRGGRLRGIRLKTPGGPVSAFLGIPFAEPPMGPRRFLPPEPKQPWSGVVDATTFQSVCYQYVDTLYPGFEGTEMWNPNRELSEDCLYLNVWTPYPRPTSPTPVLVWIYGGGFYSGASSLDVYDGRFLVQAERTVLVSMNYRVGAFGFLALPGSREAPGNVGLLDQRLALQWVQENVAAFGGDPTSVTLFGESAGAASVGMHLLSPPSRGLFHRAVLQSGAPNGPWATVGMGEARRRATQLAHLVGCPPGGTGGNDTELVACLRTRPAQVLVNHEWHVLPQESVFRFSFVPVVDGDFLSDTPEALINAGDFHGLQVLVGVVKDEGSYFLVYGAPGFSKDNESLISRAEFLAGVRVGVPQVSDLAAEAVVLHYTDWLHPEDPARLREALSDVVGDHNVVCPVAQLAGRLAAQGARVYAYVFEHRASTLSWPLWMGVPHGYEIEFIFGIPLDPSRNYTAEEKIFAQRLMRYWANFARTGDPNEPRDPKAPQWPPYTAGAQQYVSLDLRPLEVRRGLRAQACAFWNRFLPKLLSATDTLDEAERQWKAEFHRWSSYMVHWKNQFDHYSKQDRCSDL

SEQ ID NO: 16 - protein, 667 aa, Homo sapiens
MLGLVMSCPDRTLVTKVRSHPSGNQHRPTRGGSRSFHCRRGVRPRPAALRVLPRCPAFSDAACPAAMMRPPQCLLHTPSLASPLLLLLLWLLGGGVGAEGREDAELLVTVRGGRLRGIRLKTPGGPVSAFLGIPFAEPPMGPRRFLPPEPKQPWSGVVDATTFQSVCYQYVDTLYPGFEGTEMWNPNRELSEDCLYLNVWTPYPRPTSPTPVLVWIYGGGFYSGASSLDVYDGRFLVQAERTVLVSMNYRVGAFGFLALPGSREAPGNVGLLDQRLALQWVQENVAAFGGDPTSVTLFGESAGAASVGMHLLSPPSRGLFHRAVLQSGAPNGPWATVGMGEARRRATQLAHLVGCPPGGTGGNDTELVACLRTRPAQVLVNHEWHVLPQESVFRFSFVPVVDGDFLSDTPEALINAGDFHGLQVLVGVVKDEGSYFLVYGAPGFSKDNESLISRAEFLAGVRVGVPQVSDLAAEAVVLHYTDWLHPEDPARLREALSDVVGDHNVVCPVAQLAGRLAAQGARVYAYVFEHRASTLSWPLWMGVPHGYEIEFIFGIPLDPSRNYTAEEKIFAQRLMRYWANFARTGDPNEPRDPKAPQWPPYTAGAQQYVSLDLRPLEVRRGLRAQACAFWNRFLPKLLSATGMQGPAGSGWEEGSGSPPGVTPLFSP

SEQ ID NO: 17 - protein, 684 aa, Homo sapiens
MLGLVMSCPDRTLVTKVRSHPSGNQHRPTRGGSRSFHCRRGVRPRPAALRVLPRCPAFSDAACPAAMMRPPQCLLHTPSLASPLLLLLLWLLGGGVGAEGREDAELLVTVRGGRLRGIRLKTPGGPVSAFLGIPFAEPPMGPRRFLPPEPKQPWSGVVDATTFQSVCYQYVDTLYPGFEGTEMWNPNRELSEDCLYLNVWTPYPRPTSPTPVLVWIYGGGFYSGASSLDVYDGRFLVQAERTVLVSMNYRVGAFGFLALPGSREAPGNVGLLDQRLALQWVQENVAAFGGDPTSVTLFGESAGAASVGMHLLSPPSRGLFHRAVLQSGAPNGPWATVGMGEARRRATQLAHLVGCPPGGTGGNDTELVACLRTRPAQVLVNHEWHVLPQESVFRFSFVPVVDGDFLSDTPEALINAGDFHGLQVLVGVVKDEGSYFLVYGAPGFSKDNESLISRAEFLAGVRVGVPQVSDLAAEAVVLHYTDWLHPEDPARLREALSDVVGDHNVVCPVAQLAGRLAAQGARVYAYVFEHRASTLSWPLWMGVPHGYEIEFIFGIPLDPSRNYTAEEKIFAQRLMRYWANFARTGDPNEPRDPKAPQWPPYTAGAQQYVSLDLRPLEVRRGLRAQACAFWNRFLPKLLSATASEAPSTCPGFTHGEAAPRPGLPLPLLLLHQLLLLFLSHLRRL

SEQ ID NO: 18 - RNA, 40 nt, Mus musculus
cuggugucagaacucaagccccuauugcauccccauauug

SEQ ID NO: 19 - RNA, 48 nt, Mus musculus
ugugugacagacggaccgcagccugcggagacaccagacaccguucac

SEQ ID NO: 20 - RNA, 45 nt, Mus musculus
ucgucaccaggguccggucggggcaugacaucaccaggccuagca

SEQ ID NO: 21 - DNA, 19 bp, Mus musculus
agcggagggcattgcaata

SEQ ID NO: 22 - DNA, 22 bp, Mus musculus
tttgatctcttggctggagacg

SEQ ID NO: 23 - DNA, 21 bp, Mus musculus
ggaacattggccgcctccagc

SEQ ID NO: 24 - DNA, 21 bp, Mus musculus
caggctgcggtccgtctgtca

SEQ ID NO: 25 - DNA, 19 bp, Homo sapiens
cctggtgacgaaagtccga

SEQ ID NO: 26 - DNA, 25 bp, Mus musculus
ccgggtctatgcctacatctttgaa

SEQ ID NO: 27 - DNA, 18 bp, Mus musculus
cgggtctatgcctacatc

SEQ ID NO: 28 - DNA, 25 bp, Mus musculus
ccgggtctatgcctacatctttgaa

SEQ ID NO: 29 - DNA, 20 bp, Mus musculus
ccagcagctgcgggtcttcc

SEQ ID NO: 30 - DNA, 21 bp, Homo sapiens
tcctccacccaggagccagag

SEQ ID NO: 31 - DNA, 28 bp, Mus musculus
aaggaagaagaggagggacagggctaag

SEQ ID NO: 32 - DNA, 21 bp, Mus musculus
gctcggtcgtattatatccca

SEQ ID NO: 33 - protein, 31 aa, Mus musculus
MRPPWYPLHTPSLAFPLLFLLLSLLGGGARA

SEQ ID NO: 34 - protein, 31 aa, Homo sapiens
MRPPQCLLHTPSLASPLLLLLLWLLGGGVGA

SEQ ID NO: 35 - RNA, 48 nt, Artificial Sequence
  mE1b probe
cuccccgcccgagccuugguguggggguaucuggagaaucgugagcau

SEQ ID NO: 36 - DNA, 2270 bp, Homo sapiens
gggtctcgggggctcctgagccggccgcctccagcggagggagcaggcgccgcgcggtattgccgcatgcacctcggtcgtggggaccccgctgcagcagccctgtccacacgggtgaactcctggagggcagggcggggggcaatctgcgcgggtcaggcccctcggagcaggctgggggcgccgagccaggccgctcccacctgccagccgcgtggccaatgaatgctaggcctggtgatgtcatgccccgaccggaccctggtgacgaaagtccgaagtcacccgtcagggaaccagcacagacccacccgcgggggttccagaagtttccactgccgccgcggagtgcggcctcgcccagcagccttgcgcgtgctaccacgctgtcctgccttctcagcagacgccgcctgccctgcagccatgaggcccccgcagtgtctgctgcacacgccttccctggcttccccactccttctcctcctcctctggctcctgggtggaggagtgggggctgagggccgggaggatgcagagctgctggtgacggtgcgtgggggccggctgcggggcattcgcctgaagacccccgggggccctgtctctgctttcctgggcatcccctttgcggagccacccatgggaccccgtcgctttctgccaccggagcccaagcagccttggtcaggggtggtagacgctacaaccttccagagtgtctgctaccaatatgtggacaccctatacccaggttttgagggcaccgagatgtggaaccccaaccgtgagctgagcgaggactgcctgtacctcaacgtgtggacaccatacccccggcctacatcccccacccctgtcctcgtctggatctatgggggtggcttctacagtggggcctcctccttggacgtgtacgatggccgcttcttggtacaggccgagaggactgtgctggtgtccatgaactaccgggtgggagcctttggcttcctggccctgccggggagccgagaggccccgggcaatgtgggtctcctggatcagaggctggccctgcagtgggtgcaggagaacgtggcagccttcgggggtgacccgacatcagtgacgctgtttggggagagcgcgggagccgcctcggtgggcatgcacctgctgtccccgcccagccggggcctgttccacagggccgtgctgcagagcggtgcccccaatggaccctgggccacggtgggcatgggagaggcccgtcgcagggccacgcagctggcccaccttgtgggctgtcctccaggcggcactggtgggaatgacacagagctggtagcctgccttcggacacgaccagcgcaggtcctggtgaaccacgaatggcacgtgctgcctcaagaaagcgtcttccggttctccttcgtgcctgtggtagatggagacttcctcagtgacaccccagaggccctcatcaacgcgggagacttccacggcctgcaggtgctggtgggtgtggtgaaggatgagggctcgtattttctggtttacggggccccaggcttcagcaaagacaacgagtctctcatcagccgggccgagttcctggccggggtgcgggtcggggttccccaggtaagtgacctggcagccgaggctgtggtcctgcattacacagactggctgcatcccgaggacccggcacgcctgagggaggccctgagcgatgtggtgggcgaccacaatgtcgtgtgccccgtggcccagctggctgggcgactggctgcccagggtgcccgggtctaccctacgtctttgaacaccgtgcttccacgctctcctggcccctgtggatgggggtgccccacggctacgagatcgagttcatctttgggatccccctggacccctctcgaaactacacggcagaggagaaaatcttcgcccagcgactgatgcgatactgggccaactttgcccgcacaggggatcccaatgagccccgagaccccaaggccccacaatggcccccgtacacggcgggggctcagcagtacgttagtctggacctgcggccgctggaggtgcggcgggggctgcgcgcccaggcctgcgccttctggaaccgcttcctccccaaattgctcagcgccaccgacacgctcgacgaggcggagcgccagtggaaggccgagttccaccgctggagctcctacatggtgcactggaagaaccagttcgaccactacagcaagcaggatcgctgctcagacctgtga

SEQ ID NO: 37 - DNA, 2359 bp, Homo sapiens
gggtctcgggggctcctgagccggccgcctccagcggagggagcaggcgccgcgcggtattgccgcatgcacctcggtcgtggggaccccgctgcagcagccctgtccacacgggtgaactcctggagggcagggcggggggcaatctgcgcgggtcaggcccctcggagcaggctgggggcgccgagccaggccgctcccacctgccagccgcgtggccaatgaatgctaggcctggtgatgtcatgccccgaccggaccctggtgacgaaagtccgaagtcacccgtcagggaaccagcacagacccacccgcgggggttccagaagtttccactgccgccgcggagtgcggcctcgcccagcagccttgcgcgtgctaccacgctgtcctgccttctcagcagacgccgcctgccctgcagccatgaggcccccgcagtgtctgctgcacacgccttccctggcttccccactccttctcctcctcctctggctcctgggtggaggagtgggggctgagggccgggaggatgcagagctgctggtgacggtgcgtgggggccggctgcggggcattcgcctgaagacccccgggggccctgtctctgctttcctgggcatcccctttgcggagccacccatgggaccccgtcgctttctgccaccggagcccaagcagccttggtcaggggtggtagacgctacaaccttccagagtgtctgctaccaatatgtggacaccctatacccaggttttgagggcaccgagatgtggaaccccaaccgtgagctgagcgaggactgcctgtacctcaacgtgtggacaccatacccccggcctacatcccccacccctgtcctcgtctggatctatgggggtggcttctacagtggggcctcctccttggacgtgtacgatggccgcttcttggtacaggccgagaggactgtgctggtgtccatgaactaccgggtgggagcctttggcttcctggccctgccggggagccgagaggccccgggcaatgtgggtctcctggatcagaggctggccctgcagtgggtgcaggagaacgtggcagccttcgggggtgacccgacatcagtgacgctgtttggggagagcgcgggagccgcctcggtgggcatgcacctgctgtccccgcccagccggggcctgttccacagggccgtgctgcagagcggtgcccccaatggaccctgggccacggtgggcatgggagaggcccgtcgcagggccacgcagctggcccaccttgtgggctgtcctccaggcggcactggtgggaatgacacagagctggtagcctgccttcggacacgaccagcgcaggtcctggtgaaccacgaatggcacgtgctgcctcaagaaagcgtcttccggttctccttcgtgcctgtggtagatggagacttcctcagtgacaccccagaggccctcatcaacgcgggagacttccacggcctgcaggtgctggtgggtgtggtgaaggatgagggctcgtattttctggtttacggggccccaggcttcagcaaagacaacgagtctctcatcagccgggccgagttcctggccggggtgcgggtcggggttccccaggtaagtgacctggcagccgaggctgtggtcctgcattacacagactggctgcatcccgaggacccggcacgcctgagggaggccctgagcgatgtggtgggcgaccacaatgtcgtgtgccccgtggcccagctggctgggcgactggctgcccagggtgcccgggtctaccctacgtctttgaacaccgtgcttccacgctctcctggcccctgtggatgggggtgccccacggctacgagatcgagttcatctttgggatccccctggacccctctcgaaactacacggcagaggagaaaatcttcgcccagcgactgatgcgatactgggccaactttgcccgcacaggggatcccaatgagccccgagaccccaaggccccacaatggcccccgtacacggcgggggctcagcagtacgttagtctggacctgcggccgctggaggtgcggcgggggctgcgcgcccaggcctgcgccttctggaaccgcttcctccccaaattgctcagcgccaccggtatgcaggggccagcgggcagcggctgggaggaggggagtgggagcccgccaggtgtaacccctctcttctccccctagcctcggaggctcccagcacctgcccaggcttcacccatggggaggctgctcggaggcccggcctccccctgcccctcctcctcctccaccagcttctcctcctcttcctctcccacctccggcggctgtga

SEQ ID NO: 38 - DNA, 3240 bp, Homo sapiens
gggtctcgggggctcctgagccggccgcctccagcggagggagcaggcgccgcgcggtattgccgcatgcacctcggtcgtggggaccccgctgcagcagccctgtccacacgggtgaactcctggagggcagggcggggggcaatctgcgcgggtcaggcccctcggagcaggctgggggcgccgagccaggccgctcccacctgccagccgcgtggccaatgaatgctaggcctggtgatgtcatgccccgaccggaccctggtgacgaaagtccgaagtcacccgtcagggaaccagcacagacccacccgcgggggttccagaagtttccactgccgccgcggagtgcggcctcgcccagcagccttgcgcgtgctaccacgctgtcctgccttctcacagacgccgcctgccctgcagccatgaggcccccgcagtgtctgctgcacacgccttccctggcttccccactccttctcctcctcctctggctcctgggtggaggagtgggggctgagggccgggaggatgcagagctgctggtgacggtgcgtgggggccggctgcggggcattcgcctgaagacccccgggggccctgtctctgctttcctgggcatcccctttgcggagccacccatgggaccccgtcgctttctgccaccggagcccaagcagccttggtcaggggtggtagacgctacaaccttccagagtgtctgctaccaatatgtggacaccctatacccaggttttgagggcaccgagatgtggaaccccaaccgtgagctgagcgaggactgcctgtacctcaacgtgtggacaccatacccccggcctacatcccccacccctgtcctcgtctggatctatgggggtggcttctacagtggggcctcctccttggacgtgtacgatggccgcttcttggtacaggccgagaggactgtgctggtgtccatgaactaccgggtgggagcctttggcttcctggccctgccggggagccgagaggccccgggcaatgtgggtctcctggatcagaggctggccctgcagtgggtgcaggagaacgtggcagccttcgggggtgacccgacatcagtgacgctgtttggggagagcgcgggagccgcctcggtgggcatgcacctgctgtccccgcccagccggggcctgttccacagggccgtgctgcagagcggtgcccccaatggaccctgggccacggtgggcatgggagaggcccgtcgcagggccacgcagctggcccaccttgtgggctgtcctccaggcggcactggtgggaatgacacagagctggtagcctgccttcggacacgaccagcgcaggtcctggtgaaccacgaatggcacgtgctgcctcaagaaagcgtcttccggttctccttcgtgcctgtggtagatggagacttcctcagtgacaccccagaggccctcatcaacgcgggagacttccacggcctgcaggtgctggtgggtgtggtgaaggatgagggctcgtattttctggtttacggggccccaggcttcagcaaagacaacgagtctctcatcagccgggccgagttcctggccggggtgcgggtcggggttccccaggtaagtgacctggcagccgaggctgtggtcctgcattacacagactggctgcatcccgaggacccggcacgcctgagggaggccctgagcgatgtggtgggcgaccacaatgtcgtgtgccccgtggcccagctggctgggcgactggctgcccagggtgcccgggtctacgcctacgtctttgaacaccgtgcttccacgctctcctggcccctgtggatgggggtgccccacggctacgagatcgagttcatctttgggatccccctggacccctctcgaaactacacggcagaggagaaaatcttcgcccagcgactgatgcgatactgggccaactttgcccgcacaggggatcccaatgagccccgagaccccaaggccccacaatggcccccgtacacggcgggggctcagcagtacgttagtctggacctgcggccgctggaggtgcggcgggggctgcgcgcccaggcctgcgccttctggaaccgcttcctccccaaattgctcagcgccaccgcctcggaggctcccagcacctgcccaggcttcacccatggggaggctgctccgaggcccggcctccccctgcccctcctcctcctccaccagcttctcctcctcttcctctcccacctccggcggctgtgaacacggcctcttcccctacggccacaggggcccctcctctaatgagtggtcggaccgtggggaagggccccactcagggatctcagacctagtgctcccttcctcctcaaaccgagagactcacactggacagggcaggaggagggggccgtgcctcccacccttctcagggacccccacgcctttgttgtttgaatggaaatggaaaagccagtattcttttataaaattatcttttggaacctgagcctgacattggggggaagtgggaggccccggacggggtagcaccccccattggggctataacggtcaaccatttctgtctcttctttttcccccaacctccccctcctgtcccctctgttcccgtcttccggtcattcttttctcctcctctctccttcctgctgtccttctccggccccgcctctgccctcatcctccctctcgtctttcgcacattctcctgatcctcttgccaccgtcccacgtggtcgcctgcatttctccgtgcgtcctccctgcactgaaacccccccttcaacccgcccaaatgtccgatccccgaccttcctcgtgccgtcctcccctcccgcctcgctgggcgccctggccgcagacacgctcgacgaggcggagcgccagtggaaggccgagttccaccgctggagctcctacatggtgcactggaagaaccagttcgaccactacagcaagcaggatcgctgctcagacctgtgaccccggcgggacccccatgtcctccgctccgcccggccccctagctgtatatactatttatttcagggctgggctataacacagacgagccccagactctgcccatccccaccccaccccgacgtcccccggggctcccggtcctctggcatgtcttcaggctgagctcctccccgcgtgccttcgccctctggctgcaaataaactgttacaggcc

SEQ ID NO: 39 - protein, 8 aa, Homo sapiens
MLGLVMSC